(19) United States
(12) Patent Application Publication
(10) Pub. No.: US 2006/0018904 A1
(43) Pub. Date: Jan. 26, 2006

(54) Combination therapies utilizing benzamide inhibitors of the P2X7 receptor

(75) Inventors: James B. Chung, Thousand Oaks, CA (US); Christopher A. Gabel, Dexter, MI (US); Gail L. Jungbluth, Dexter, MI (US)

Correspondence Address: WARNER-LAMBERT COMPANY, 2800 PLYMOUTH RD, ANN ARBOR, MI, 48105, US

(73) Assignee: WARNER-LAMBERT COMPANY LLC

(21) Appl. No.: 11/168,602

(22) Filed: Jun. 28, 2005

Related U.S. Application Data
(60) Provisional application No. 60/583,943, filed on Jun. 29, 2004.

(51) Int. Cl. A61K 39/395; A61K 31/401; A61K 31/366; A61K 31/165; A61K 31/573

(52) U.S. Cl. 424/145.1; 514/171; 514/2; 514/357; 514/616; 514/620; 514/521; 514/401; 514/460; 514/548; 514/423

(57) ABSTRACT

This invention provides methods of treatment of IL-1 mediated diseases comprising administering a pharmaceutically effective amount of a pharmaceutical agent selected from the group of sulfasalazine, a statin, a glucocorticoid agent, an inhibitor of p38 kinase, an anti-IL-6-receptor antibody, anakinra, an IL-1 monoclonal antibody, an inhibitor of JAK3 protein tyrosine kinase, a M-CSF monoclonal antibody or a humanized anti-CD20 monoclonal antibody and a benzamide inhibitor of the P2X₇ receptor of the formula: 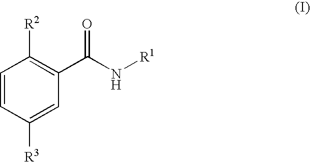
wherein R¹-R³ are as defined herein. The methods of the invention are useful in the treatment of IL-1 mediated disorders, including, without limitation, inflammatory diseases such as osteoarthritis and rheumatoid arthritis; allergies, asthma, COPD, cancer, reperfusion or ischemia in stroke or heart attack, autoimmune diseases and other disorders.

### CROSS-REFERENCE TO RELATED APPLICATIONS

This application claims priority from U.S. Provisional Patent Application No. 60/583,943 filed Jun. 29, 2004.

### FIELD OF THE INVENTION

The present invention relates to the use of benzamide inhibitors of the P2X₇ receptor in combination with other pharmaceutically or therapeutically effective agents for the treatment of IL-1 mediated conditions. The methods of the present invention are useful in the treatment of inflammatory diseases such as osteoarthritis and rheumatoid arthritis; allergies, asthma, COPD, cancer, reperfusion or ischemia in stroke or heart attack, autoimmune diseases and other disorders. The active benzamide compounds are also antagonists of the P2X₇ receptor.

### BACKGROUND OF THE INVENTION

The P2X₇ purinergic receptor (previously known as P2Z receptor), which is a ligand-gated ion channel, is present on a variety of cell types, largely those known to be involved in inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells), apoptosis, and L-selectin shedding (lymphocytes). P2X₇ receptors are also located on antigen-presenting cells (APC), keratinocytes, salivary acinar cells (parotid cells), hepatocytes and mesangial cells.

P2X₇ antagonists are known in the art, such as those described in International Patent Publications WO 01/46200, WO 01/42194, WO 01/44213, WO99/29660, WO 00/61569, WO 99/29661, WO 99/29686, WO 00/71529, and WO 01/44170, as well as in WO2003042191.

Benzamides, heteroarylamides and reverse amides for uses other than inhibition of the P2X₇ receptor are described in various publications, such as International Patent Publications WO 97/22600, EP 138,527, WO 00/71509, WO 98/28269, WO 99/17777 and WO 01/58883.

Antagonists of the P2X₇ receptor are being identified for the treatment of human disease (see e.g., Alcaraz et al. (2003) Bioorg Med Chem Lett. 13(22):4043-4046; Baxter et al. (2003) Bioorg Med Chem Lett. 13(22):4047-4050). There is a need for additional compounds, compositions, and methods of preparing compounds that can inhibit the P2X₇ receptor for use as pharmaceutical agents.

### SUMMARY OF THE INVENTION

The present invention comprises a method of treatment of an IL-1 mediated disease in a mammal, the method comprising administering to a mammal in need thereof a pharmaceutically effective amount of an inhibitor of the P2X₇ receptor and a pharmaceutically effective amount of a pharmaceutical agent selected from:
a) sulfasalazine;
b) a statin, such as atorvastatin, lovastatin, pravastatin, simvastatin, fluvastatin, cerivastatin, crilvastatin, dalvastatin, rosuvastatin, tenivastatin, fluindostatin, velostatin, dalvastatin, nisvastatin, bervastatin, pitavastatin, rivastatin, glenvastatin, eptastatin, tenivastatin, flurastatin, rosuvastatin or itavastatin;
c) a glucocorticoid agent, such as dexamethasone, methylprednisolone, prednisolone, prednisone and hydrocortisone;
d) an inhibitor of p38 kinase;
e) an anti-IL-6-receptor antibody;
f) anakinra (Kineret®);
g) an anti-IL-1 monoclonal antibody;
h) an inhibitor of JAK3 protein tyrosine kinase;
i) an anti-macrophage colony stimulation factor (M-CSF) monoclonal antibody; or
j) an anti-CD20 monoclonal antibody, such as rituximab (Rituxan®), PRO70769, HuMax-CD20 (Genmab A/S), AME-133 (Applied Molecular Evolution), or hA20 (Immunomedics, Inc.).

The methods of the present invention include the use of a P2X₇ receptor-inhibiting compound of the formula 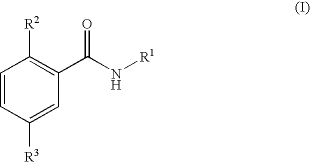
wherein R¹ is (C₁-C₆)alkyl, optionally substituted by (C₃-C₁₀)cycloalkyl, (C₆-C₁₀)aryl, (C₁-C₁₀)heterocyclyl, or (C₁-C₁₀)heteroaryl, wherein each of said (C₁-C₆)alkyl, (C₃-C₁₀)cycloalkyl, (C₆-C₁₀)aryl, (C₁-C₁₀)heterocyclyl, or (C₁-C₁₀)heteroaryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-;
R² is hydrogen, halogen, —CN, and (C₁-C₆)alkyl, wherein said (C₁-C₆)alkyl is optionally substituted by one to three suitable moieties, independently selected from the group consisting of halo, hydroxy, amino, —CN, (C₁-C₆)alkyl, (C₁-C₆)alkoxy, —CF₃, CF₃O—, (C₁-C₆)alkyl-NH—, [(C₁-C₆)alkyl]₂—N—, (C₁-C₆)alkyl-S—, (C₁-C₆)alkyl-(S═O)—, (C₁-C₆)alkyl-(SO₂)—, (C₁-C₆)alkyl-O—(C═O)—, formyl, (C₁-C₆)alkyl-(C═O)—, and (C₃-C₆)cycloalkyl; and
R³ is a suitably substituted nitrogen linked (C₁-C₁₀)heterocyclyl of the formula: 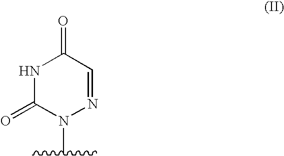
   or the pharmaceutically acceptable salts or solvates or prodrugs thereof.

IL-1 mediated diseases and disorders that my be treated with the methods and combinations of this invention include, but are not limited to, rheumatoid arthritis, osteoarthritis, juvenile arthritis, including juvenile idiopathic arthritis, Crohn's disease, chronic obstructive pulmonary disease, inflammatory bowel disease, Alzheimer's disease, psoriasis, psoriatic arthritis and atherosclerosis.

This invention also provides kits, each comprising a pharmaceutical formulation containing a pharmaceutically effective amount of a P2X₇ receptor inhibiting compound of the formula (I), or a pharmaceutically acceptable salt form thereof, and a pharmaceutical formulation containing a pharmaceutically effective amount of a pharmaceutical agent selected from sulfasalazine, a statin, a glucocorticoid agent, such as dexamethasone, methylprednisolone, prednisolone, prednisone and hydrocortisone, an inhibitor of p38 kinase, an anti-IL-6-receptor antibody, anakinra, an IL-1 monoclonal antibody, an inhibitor of JAK3 protein tyrosine kinase, a macrophage colony stimulation factor (M-CSF) monoclonal antibody, or an anti-CD20 monoclonal antibody.

### DETAILED DESCRIPTION OF THE INVENTION

It will be understood that the commercially available pharmaceutically useful agents described herein may be utilized according to the dosages and administration regimens known in the art. Examples include, but are not limited to, the dosages and regimens described in the 2004 Physicians' Desk Reference®, 58th Edition, published by Medical Economics Company, Inc. at Montvale, N.J.

Sulfasalazine is commercially available as AZULFIDINE EN-tabs® sulfasalazine delayed release tablets, USP enteric-coated tablets (Pharmacia & Upjohn) and may be administered at an initial dose of 1 to 2 g daily, with an increase as needed to 3 to 4 g daily in evenly divided doses with dosage intervals not exceeding eight hours.

Statin compounds useful with the present methods and combinations include atorvastatin, lovastatin, pravastatin, simvastatin, fluvastatin, cerivastatin, crilvastatin, dalvastatin, rosuvastatin, tenivastatin, fluindostatin, velostatin, dalvastatin, nisvastatin, bervastatin, pitavastatin, rivastatin, glenvastatin, eptastatin, tenivastatin, flurastatin, rosuvastatin and itavastatin, as well as the pharmaceutically acceptable salt forms thereof. LIPITOR® (Pfizer) (Atorvastatin Calcium) tablets may be administered at a starting dose of from 10 to 40 mg once daily. Atorvastatin calcium may be administered at a dosage range of from 10 to 80 mg once daily. Lovastatin is available as ADVICOR™ (niacin extended-release/lovastatin tablets) (Kos) and has a recommended starting dose of lovastatin of 20 mg once a day. The usual recommended starting dose for NIASPAN is 500 mg qhs and may be titrated and the dose should may be increased by not more than 500 mg every 4 weeks up to a maximum dose of 2000 mg a day. Lovastatin is also commercially available as ALTOCOR™ EXTENDED-RELEASE TABLETS (Andrx Labs) and MEVACOR® Tablets (Merck), which are recommended to have a starting dose of 20, 40, or 60 mg once a day given in the evening at bedtime and a recommended dosing range of 10-60 mg/day, in single doses. Pravastatin is commercially available as PRAVACHOL® pravastatin sodium tablets (Bristol-Myers Squibb) and PRAVIGARD™ PAC buffered aspirin and pravastatin sodium tablets (Bristol-Myers Squibb), with a recommended daily dosage range of from 10 to 80 mg given once daily. Fluvastatin is commercially available as Lescol® (fluvastatin sodium) Capsules and Lescol® XL (fluvastatin sodium) Extended-Release Tablets from Novartis Pharmaceuticals and Reliant Pharmaceuticals, with a recommended dosage range of from 20 to 80 mg/day. Simvastatin is available as ZOCOR® tablets from Merck, with a recommended starting dosage of from 20 to 40 mg/day, which may be adjusted as needed to a daily dosage range of from 5 to 80 mg/day.

Glucocorticoid agents useful in the methods and combinations herein include dexamethasone, methylprednisolone, prendisolone, prednisone and hydrocortisone. Dexamethasone is commercially available as DECADRON® Phosphate Injection (Merck) (Dexamethasone Sodium Phosphate), DECADRON® Phosphate (Merck) (Dexamethasone Sodium Phosphate) 0.05% Dexamethasone Phosphate Equivalent Sterile Ophthalmic Ointment, DECADRON® Phosphate (Merck) (Dexamethasone Sodium Phosphate) 0.1% Dexamethasone Phosphate Equivalent Sterile Ophthalmic Solution, DECADRON® Tablets (Merck) (Dexamethasone), Dexamethasone Intensol Oral Solution (concentrate) (Roxane), Dexamethasone Oral Solution (Roxane), Dexamethasone Tablets (Par), Dexamethasone Tablets (Roxane) and DEXPAK Taperpack Tablets (ECR). Dexamethasone may be administered at an initial dosage of from about 0.75 to 9 mg a day.

Methylprednisolone is commercially available as DEPO-MEDROL® (Pharmacia & Upjohn) (methylprednisolone acetate) injectable suspension, USP, DEPO-MEDROL® (Pharmacia & Upjohn) methylprednisolone acetate injectable suspension, USP, Single-Dose Vial, SOLU-MEDROL® (Pharmacia & Upjohn) methylprednisolone sodium succinate for injection, USP For Intravenous or Intramuscular Administration, and MEDROL® Tablets (Pharmacia & Upjohn). Prednisolone is available in a number of commercial forms. Predisolone acetate is available as BLEPHAMIDE® (Allergan) (sulfacetamide sodium and prednisolone acetate ophthalmic ointment, USP) 10%/0.2% sterile, BLEPHAMIDE® (Allergan) (sulfacetamide sodium-prednisolone acetate ophthalmic suspension), PRED FORTE® (Allergan) (prednisolone acetate ophthalmic suspension, USP) 1% sterile and PRED-G® (Allergan) (gentamicin and prednisolone acetate ophthalmic ointment, USP) 0.3%/0.6% sterile. Prednisolone sodium phosphate is available as ORAPRED® (Ascent) (prednisolone sodium phosphate oral solution) and PEDIAPRED® (Celltech) (prednisolone sodium phosphate, USP) Oral Solution. Hydrocortisone is available from numerous commercial sources in the form of hydrocortisone, hydrocortisone acetate, hydrocortisone butyrate and hydrocortisone sodium phosphate. Commercially available products include HYDROCORTONE® Tablets (Merck) (Hydrocortisone), PROCTOCORT® CREAM (Monarch) (hydrocortisone cream USP) 1%, and PROCTOCORT® (Monarch) (Hydrocortisone Acetate) Rectal Suppositories.

Inhibitors of p38 kinase that may be utilized in the methods and combinations of this invention include those disclosed in WO 98/52940, U.S. Pat. No. 6,514,977, WO 00/31063, U.S. Pat. No. 6,525,059, U.S. Pat. No. 6,423,713, U.S. Pat. No. 6,617,324, U.S. Pat. No. 5,932,576, WO 98/52937, U.S. Pat. No. 6,087,496, U.S. Pat. No. 6,335,336, U.S. Pat. No. 6,579,873, U.S. Pat. No. 6,087,381, WO 98/52941, U.S. Pat. No. 6,503,930, U.S. Pat. No. 6,509,361, WO 99/58523, U.S. Pat. No. 6,143,892, WO 00/31072, U.S. Pat. No. 6,242,612, U.S. Pat. No. 6,342,608, U.S. Pat. No. 6,482,955, WO 03/026663, WO 03/068,230A1, EP 1247810, WO 02/072576, WO 02/072579, U.S. 20040044002 A1, U.S. Pat. No. 6,635,644, U.S. Pat. No. 6,632,945, U.S. Pat. No. 6,608,060, U.S. Pat. No. 6,552,019, U.S. Pat. No. 6,528,508, U.S. Pat. No. 6,509,363, U.S. Pat. No. 6,509,361, U.S. Pat. No. 6,476,031, and U.S. Pat. No. 5,945,418, the contents of which are incorporated herein by reference. Specific p38 kinase inhibiting compounds that may be used include:
3-[3-bromo-4-(2,4-difluoro-benzyloxy)-6-methyl-2-oxo-2H-pyridin-1-yl]-4,N-dimethyl-benzamide;
3-[5-chloro-4-(2,4-difluoro-benzyloxy)-6-oxo-6H-pyrimidin-1-yl]-N-(2-hydroxy-ethyl)-4-methyl-benzamide;
3-[5-bromo-4-(2,4-difluoro-benzyloxy)-6-oxo-6H-pyrimidin-1-yl]-N-(2-hydroxy-1-methyl-ethyl)-4-methyl-benzamide;
3-[5-chloro-4-(2,4-difluoro-benzyloxy)-6-oxo-6H-pyrimidin-1-yl]-N-(2-hydroxy-1-methyl-ethyl)-4-methyl-benzamide;
1-{4-[5-(4-chloro-2-fluoro-phenyl)-4-pyrimidin-4-yl-2H-pyrazol-3-yl]-piperidin-1-yl}-2-hydroxy-ethanone;
1-{4-[5-(4-chloro-phenyl)-4-pyrimidin-4-yl-2H-pyrazol-3-yl]-piperidin-1-yl}-2-hydroxy-ethanone;
6-[4-(2,5-difluoro-phenyl)-oxazol-5-yl]-3-isopropyl-[1,2,4]triazolo[4,3-a]pyridine; or
3-tert-butyl-6-[4-(2,4,5-trifluoro-phenyl)-oxazol-5-yl]-[1,2,4]triazolo[4,3-a]pyridine.

An “anti-IL-6-receptor antibody” is an antibody that specifically binds the extracellular domain of an IL-6 receptor polypeptide. An example of an “anti-IL-6 receptor antibody” is Atlizumab (also known as tocilizumab, rhPM-1, MRA and R-1569) (Chugai Biopharmaceuticals, Inc.), which is a humanized monoclonal antibody to the human IL-6 receptor that was constructed by grafting the complementary determining regions (CDR) form mouse PM-1 (a specific monoclonal antibody against the human IL-6 receptor) into human IgG. Atlizumab has been administered to humans at a rate of from 0.1 to 10 mg/kg i.v in the treatment of rheumatoid arthritis. This includes rates of 2, 4 or 8 mg/kg every two weeks for 6 months. Atlizumab has also been administered to human recipients at a rate of 50 and 100 mg i.v. once or twice monthly for 5-42 weeks in a study of Castleman's disease patients (see Drugs of the Future 2003, 28(4): 315-319).

KINERET® (anakinra) (available from Amgen) may also be administered in dosages and regimens known in the art. For example anakinra for the treatment of patients with rheumatoid arthritis may be administered at a rate of 100 mg/day by subcutaneous injection.

Inhibitors of JAK3 kinase that may be utilized in the methods and combinations of this invention include those disclosed in WO 99/65908 (Blumenkopf et al.), WO 99/65909 (Blumenkopf et al.) and WO 02/00661.

RITUXAN® (rituximab) may be administered in the methods of this invention using dosages and regimens known in the art. For instance, RITUXAN® may be administered intravenously at an initial rate of 50 mg/hr and increased up to a rate of 400 mg/hr, as tolerated. Rituximab may be given at 375 mg/m ² IV infusion once weekly for 4 or 8 doses. Rituximab has also been given in two injections of 1,000 mg fifteen days apart in the treatment of rheumatoid arthritis (see New England Journal of Medicine 2004, Jun. 17; 350(25):2546-8).

The methods of the present invention may also utilize a P2X₇ receptor inhibiting compound of the formula 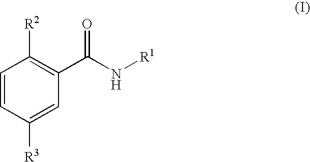
wherein R¹ is (C₁-C₆)alkyl, optionally substituted by (C₃-C₁₀)cycloalkyl, (C₆-C₁₀)aryl, (C₁-C₁₀)heterocyclyl, or (C₁-C₁₀)heteroaryl, wherein each of said (C₁-C₆)alkyl, (C₃-C₁₀)cycloalkyl, (C₆-C₁₀)aryl, (C₁-C₁₀)heterocyclyl, or (C₁-C₁₀)heteroaryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-;
R² is hydrogen, halogen, —CN, and (C₁-C₆)alkyl, wherein said (C₁-C₆)alkyl is optionally substituted by one to three suitable moieties, independently selected from the group consisting of halo, hydroxy, amino, —CN, (C₁-C₆)alkyl, (C₁-C₆)alkoxy, —CF₃, CF₃O—, (C₁-C₆)alkyl-NH—, [(C₁-C₆)alkyl]₂—N—, (C₁-C₆)alkyl-S—, (C₁-C₆)alkyl-(S═O)—, (C₁-C₆)alkyl-(SO₂)—, (C₁-C₆)alkyl-O—(C═O)—, formyl, (C₁-C₆)alkyl-(C═O)—, and (C₃-C₆)cycloalkyl;
R³ is a nitrogen linked (C₁-C₁₀)heterocyclyl of the formula: 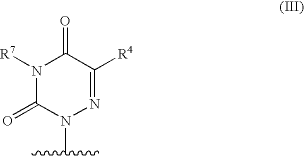
   wherein R⁴ is selected from the group of suitable substituents, such as hydrogen, halo, hydroxy, —CN, HO—(C₁-C₆)alkyl, (C₁-C₆)alkyl optionally substituted with one to three fluoro, (C₁-C₆)alkoxy optionally substituted with one to three fluoro, HO₂C—, (C₁-C₆)alkyl-O—(C═O)—, R⁵R⁶N(O₂S)—, (C₁-C₆)alkyl-(O₂S)—NH—, (C₁-C₆)alkyl-O₂S—[(C₁-C₆)alkyl-N]-, R⁵R⁶N(C═O)—, R⁵R⁶N(CH₂)ₘ—, (C₆-C₁₀)aryl, (C₃-C₈)cycloalkyl, (C₁-C₁₀)heteroaryl, (C₁-C₁₀)heterocyclyl, (C₆-C₁₀)aryl-O—, (C₃-C₈)cycloalkyl-O—, (C₁-C₁₀)heteroaryl-O— and (C₁-C₁₀)heterocyclyl-O—; and
R⁷ is selected from the group of suitable substituents such as hydrogen and (C₁-C₆)alkyl optionally substituted with one to three halogens, hydroxy, —CN, (C₁-C₆)alkoxy-, (C₂-C₆)alkenoxy, (C₁-C₆)alkyl-SO₂—, NH₂—, ((C₁-C₆)alkyl)ₙ-N—, ((C₂-C₆)alkenyl)ₙ-N—, ((C₂-C₆)alkynyl)ₙ-N—, NH₂(C═O)—, (C₁-C₆)alkyl-(C═O)N—, ((C₁-C₆)alkyl)ₙ-N—(C═O)—, (C₂-C₆)alkenyl-(C═O)N—, ((C₂-C₆)alkenyl)ₙ-N—(C═O)—, (C₂-C₆)alkynyl-(C═O)N—, ((C₂-C₆)alkynyl)ₙ-N—(C═O)—, (C₁-C₆)alkyl-(C═O)—, (C₂-C₆)alkenyl-(C═O)—, (C₂-C₆)alkynyl-(C═O)—, (C₃-C₁₀)cycloalkyl-(C═O)—, ((C₁-C₁₀)heterocyclyl-(C═O)—, (C₆-C₁₀)aryl-(C═O), (C₁-C₁₀)heteroaryl-(C═O), (C₁-C₆)alkyl-(C═O)O—, (C₂-C₆)alkenyl-(C═O)O—, (C₂-C₆)alkynyl-(C═O)O—, (C₁-C₆)alkyl-O(C═O)—, (C₂-C₆)alkenyl-O—(C═O)—, (C₂-C₆)alkynyl-O—(C═O)—, (C₃-C₁₀)cycloalkyl, (C₆-C₁₀)aryl, (C₁-C₁₀)heterocyclyl, and (C₁-C₁₀)heteroaryl;
wherein R⁴ and R⁷ may each be optionally substituted on any aliphatic or aromatic carbon atom by one to three suitable moieties, independently selected from the group consisting of halo, hydroxy, amino, —CN, (C₁-C₆)alkyl, (C₁-C₆)alkoxy, —CF₃, CF₃O—, (C₁-C₆)alkyl-NH—, [(C₁-C₆)alkyl]₂—N—, (C₁-C₆)alkyl-S—, (C₁-C₆)alkyl-(S═O)—, (C₁-C₆)alkyl-(SO₂)—, (C₁-C₆)alkyl-O—(C═O)—, formyl, (C₁-C₆)alkyl-(C═O)—, and (C₃-C₆)cycloalkyl;
   R⁵ and R⁶ are each independently selected from the group consisting of hydrogen, (C₁-C₆)alkyl, HO—(C₂-C₆)alkyl- and (C₃-C₈)cycloalkyl, or R⁵ and R⁶ may optionally be taken together with the nitrogen atom to which they are attached to form a 3 to 8 membered heterocycle;
   n is an integer from zero to two; and
   m is an integer from one to two;
      or the pharmaceutically acceptable salts or solvates or prodrugs thereof.

The methods of treatment, pharmaceutical combinations and pharmaceutical compositions of this invention also include the use of compounds of the formula: 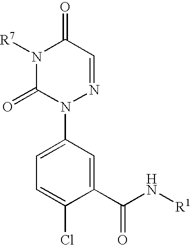
wherein R⁷ is as defined herein and R¹ is C₁-C₃ alkyl substituted by a C₃-C₈ or phenyl ring, the C₃-C₈ and phenyl rings being optionally substituted by from 1 to 4 substituents selected from the group of OH, halo, C₁-C₃ alkyl, C₁-C₃ alkoxy, or C₁-C₃ alkyl substituted by OH.

This invention includes a method of treatment of rheumatoid arthritis in a mammal, the method comprising administering to the mammal a pharmaceutically effective amount of sulfasalazine, or a pharmaceutically acceptable salt thereof, and a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide. This invention also includes a pharmaceutical composition comprising a pharmaceutically effective amount of sulfasalazine, or a pharmaceutically acceptable salt thereof, a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide and one or more pharmaceutically acceptable carriers or excipients. This invention further comprises a kit comprising a pharmaceutical formulation containing a pharmaceutically effective amount of sulfasalazine, or a pharmaceutically acceptable salt form thereof, and a pharmaceutical formulation containing a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide.

This invention includes a method of treatment of rheumatoid arthritis in a mammal, the method comprising administering to a mammal in need thereof a pharmaceutically effective amount of atorvastatin, or a pharmaceutically acceptable salt form thereof, and a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide. This invention also includes a pharmaceutical composition comprising a pharmaceutically effective amount of atorvastatin, or a pharmaceutically acceptable salt form thereof, a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide and one or more pharmaceutically acceptable carriers or excipients. This invention further comprises a kit comprising a pharmaceutical formulation containing a pharmaceutically effective amount of atorvastatin, or a pharmaceutically acceptable salt form thereof, and a pharmaceutical formulation containing a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide. Among the useful salt forms of atorvastatin in the methods, formulations and kits of this invention is atorvastatin calcium.

The present methods may also utilize the pharmaceutically acceptable acid addition salts of compounds of the formula I. The acids which are used to prepare the pharmaceutically acceptable acid addition salts of the aforementioned base compounds of this invention are those which form non-toxic acid addition salts, i.e., salts containing pharmacologically acceptable anions, such as the chloride, bromide, iodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)]salts.

The P2X₇ inhibiting compounds of this invention also include base addition salts of formula I. The chemical bases that may be used as reagents to prepare pharmaceutically acceptable base salts of those compounds of formula I that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

This invention also encompasses pharmaceutical compositions containing prodrugs of compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain.

This invention's methods also encompass use of compounds of formula I containing protective groups. One skilled in the art will also appreciate that compounds of the invention can also be prepared with certain protecting groups that are useful for purification or storage and can be removed before administration to a patient. The protection and deprotection of functional groups is described in “Protective Groups in Organic Chemistry”, edited by J. W. F. McOmie, Plenum Press (1973) and “Protective Groups in Organic Synthesis”, 3rd edition, T. W. Greene and P. G. M. Wuts, Wiley-Interscience (1999).

The P2X₇ receptor inhibiting compounds useful in the present invention include all stereoisomers (e.g., cis and trans isomers) and all optical isomers of compounds of the formula I (e.g., R and S enantiomers), as well as racemic, diastereomeric and other mixtures of such isomers.

The P2X₇ receptor inhibiting compounds, salts and prodrugs of the present invention can exist in several tautomeric forms, including the enol and imine form, and the keto and enamine form and geometric isomers and mixtures thereof. All such tautomeric forms are included within the scope of the present invention. Tautomers exist as mixtures of a tautomeric set in solution. In solid form, usually one tautomer predominates. Even though one tautomer may be described, the present invention includes all tautomers of the present compounds. One example of a tautomeric structure is when R³ is a group of the formula 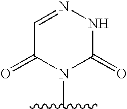
One skilled in the art will appreciate that this group can also be drawn as its tautomer

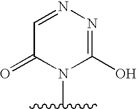

The present invention also includes atropisomers of the compounds useful in the present methods. Atropisomers refer to compounds of formula I that can be separated into rotationally restricted isomers.

The P2X₇ inhibiting compounds useful this invention may contain olefin-like double bonds. When such bonds are present, the compounds of the invention exist as cis and trans configurations and as mixtures thereof.

A “suitable substituent” is intended to mean a chemically and pharmaceutically acceptable functional group i.e., a moiety that does not negate the biological activity of the inventive compounds. Such suitable substituents may be routinely selected by those skilled in the art. Illustrative examples of suitable substituents include, but are not limited to halo groups, perfluoroalkyl groups, perfluoroalkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, amino groups, alkyl- and dialkylamino groups, carbamoyl groups, alkylcarbonyl groups, alkoxycarbonyl groups, alkylaminocarbonyl groups dialkylamino carbonyl groups, arylcarbonyl groups, aryloxycarbonyl groups, alkylsulfonyl groups, arylsulfonyl groups and the like. Those skilled in the art will appreciate that many substituents can be substituted by additional substituents. Further examples of suitable substituents include those recited in the definition of compounds of Formula I, including R¹ through R⁷, as defined hereinabove.

As used herein, the term “alkyl,” as well as the alkyl moieties of other groups referred to herein (e.g., alkoxy), may be linear or branched (such as methyl, ethyl, n-propyl, isopropyl, n-butyl, iso-butyl, secondary-butyl, tertiary-butyl); optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. The phrase “each of said alkyl” as used herein refers to any of the preceding alkyl moieties within a group such alkoxy, alkenyl or alkylamino. Preferred alkyls include (C₁-C₆)alkyl, more preferred are (C₁-C₄)alkyl, and most preferred are methyl and ethyl.

As used herein, the term “cycloalkyl” refers to a mono, bicyclic or tricyclic carbocyclic ring (e.g., cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, etc.); optionally containing 1 or 2 double bonds and optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “halogen” includes fluorine, chlorine, bromine or iodine.

As used herein, the term “alkenyl” means straight or branched chain unsaturated radicals of 2 to 6 carbon atoms, including, but not limited to ethenyl, 1-propenyl, 2-propenyl (allyl), iso-propenyl, 2-methyl-1-propenyl, 1-butenyl, 2-butenyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “alkynyl” is used herein to mean straight or branched hydrocarbon chain radicals having one triple bond including, but not limited to, ethynyl, propynyl, butynyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl.

As used herein, the term “carbonyl” or “(C═O)” (as used in phrases such as alkylcarbonyl, alkyl-(C═O)— or alkoxycarbonyl) refers to the joinder of the >C═O moiety to a second moiety such as an alkyl or amino group (i.e. an amido group). Alkoxycarbonylamino (i.e. alkoxy(C═O)—NH—) refers to an alkyl carbamate group.

The carbonyl group is also equivalently defined herein as (C═O). Alkylcarbonylamino refers to groups such as acetamide.

As used herein, the term “oxo” is used herein to mean a double bonded oxygen (═O) radical wherein the bond partner is a carbon atom. Such a radical can also be thought as a carbonyl group.

As used herein, the term “(C₁-C₄)alkyl-O₂S—[(C₁-C₄)alkyl-N]—” is used to mean a radical of the formula

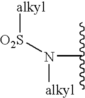

As used herein, the term “aryl” means aromatic radicals such as phenyl, naphthyl, tetrahydronaphthyl, indanyl and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. As used herein, the term “heteroaryl” refers to an aromatic heterocyclic group usually with one heteroatom selected from O, S and N in the ring. In addition to said heteroatom, the aromatic group may optionally have up to four N atoms in the ring. For example, heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like; optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. Particularly preferred heteroaryl groups include oxazolyl, imidazolyl, pyridyl, thienyl, furyl, thiazolyl and pyrazolyl.

The term “heterocyclic” as used herein refers to a cyclic group containing 1-9 carbon atoms and 1 to 4 hetero atoms selected from N, O, S(O)ₙ or NR. Examples of such rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, and the like. Examples of said monocyclic saturated or partially saturated ring systems are tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholin-yl, 1,2-tetrahydrothiazin-2-yl, 1,3-tetrahydrothiazin-3-yl, tetrahydrothiadiazin-yl, morpholin-yl, 1,2-tetrahydrodiazin-2-yl, 1,3-tetrahydrodiazin-1-yl, 1,4-oxazin-2-yl, 1,2,5-oxathiazin-4-yl and the like; optionally containing 1 or 2 double bonds and optionally substituted by 1 to 3 suitable substituents as defined above such as fluoro, chloro, trifluoromethyl, (C₁-C₆)alkoxy, (C₆-C₁₀)aryloxy, trifluoromethoxy, difluoromethoxy or (C₁-C₆)alkyl. Preferred heterocyclics include tetrahydrofuranyl, pyrrolidinyl, piperidinyl, piperazinyl and morpholinyl.

Nitrogen heteroatoms as used herein refers to N═, >N and —NH; wherein —N=refers to a nitrogen double bond; >N refers to a nitrogen containing two bond connections and —N refers to a nitrogen containing one bond.

“Embodiment” as used herein refers to specific groupings of compounds or uses into discrete subgenera. Such subgenera may be cognizable according to one particular substituent such as a specific R¹ or R³ group. Other subgenera are cognizable according to combinations of various substituents, such as all compounds wherein R² is chloro and R¹ is (C₁-C₄)alkyl, optionally substituted by (C₃-C₁₀)cycloalkyl. The phrase “in combination with each of the aforementioned embodiments” refers to combinations of the identified embodiment with each embodiment previously identified in the specification. Thus an embodiment of compounds wherein R¹ is (C₁-C₄)alkyl, optionally substituted by (C₃-C₁₀)cycloalkyl “in combination with each of the aforementioned embodiments” refers to additional embodiments comprising combinations with each embodiment previously identified in the specification.

Thus, the invention provides the use of compounds in which R¹ is (C₁-C₄)alkyl, optionally substituted by (C₃-C₁₀)cycloalkyl; wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The invention further provides compounds in which R¹ is (C₁-C₄)alkyl, optionally substituted by (C₆-C₁₀)aryl; wherein said (C₁-C₄)alkyl or (C₆-C₁₀)aryl are optionally substituted by one to three suitable moieties independently selected from the group consisting of hydroxy, halogen, CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Moreover, the invention contemplates compounds in which R is halogen and (C₁-C₆)alkyl, and preferably compounds in which R² is chloro and methyl or ethyl.

In one embodiment of the invention, R³ is a nitrogen linked (C₁-C₁₀)heterocyclyl of formula (III), wherein R⁴ is hydrogen and R⁷ is independently selected from the group of suitable substituents such as hydrogen and (C₁-C₆)alkyl, wherein said (C₁-C₆)alkyl is optionally substituted with one to three substituents independently selected from halo, hydroxy, —CN, (C₁-C₆)alkoxy-, (C₂-C₆)alkenoxy, (C₁-C₆)alkyl-SO₂—, NH₂—, (C₁-C₆)alkyl)ₙ-N—, ((C₂-C₆)alkenyl), —N—, ((C₂-C₆)alkynyl)ₙ-N—, NH₂(C═O)-, (C₁-C₆)alkyl-(C═O)N—, ((C₁-C₆)alkyl)ₙ-N—(C═O)—, (C₂-C₆)alkenyl-(C═O)N—, ((C₂-C₆)alkenyl), —N—(C═O)—, (C₂-C₆)alkynyl-(C═O)N—, ((C₂-C₆)alkynyl)ₙ-N—(C═O)—, (C₁-C₆)alkyl-(C═O)—, (C₂-C₆)alkenyl-(C═O)—, (C₂-C₆)alkynyl-(C═O)—, (C₃-C₁₀)cycloalkyl-(C═O)—, ((C₁-C₁₀)heterocyclyl-(C═O)—, (C₆-C₁₀)aryl-(C═O), (C₁-C₁₀)heteroaryl-(C═O), (C₁-C₆)alkyl-(C═O)O—, (C₂-C₆)alkenyl-(C═O)O—, (C₂-C₆)alkynyl-(C═O)O—, (C₁-C₆)alkyl-O(C═O)—, (C₂-C₆)alkenyl-O—(C═O)—, (C₂-C₆)alkynyl-O—(C═O)—, (C₃-C₁₀)cycloalkyl, (C₆-C₁₀)aryl, (C₁-C₁₀)heterocyclyl, and (C₁-C₁₀)heteroaryl; wherein R⁷ may optionally be substituted on any ring aliphatic or aromatic carbon atom by one to three suitable moieties, independently selected from the group consisting of halo, hydroxy, amino, —CN, (C₁-C₄)alkyl, (C₁-C₄)alkoxy, —CF₃, CF₃O—, (C₁-C₄)alkyl-NH—, [(C₁-C₄)alkyl]₂—N—, (C₁-C₄)alkyl-S—, (C₁-C₄)alkyl-(S═O)—, (C₁-C₄)alkyl-(SO₂)—, (C₁-C₄)alkyl-O—(C═O)—, formyl, (C₁-C₄)alkyl-(C═O)—, and (C₃-C₆)cycloalkyl.

Another embodiment of the invention is compounds in which R⁷ is hydrogen.

A further embodiment of the invention are compounds in which R³ is a nitrogen linked (C₁-C₁₀)heterocyclyl of formula (III), wherein R⁴ is hydrogen and R⁷ is (C₁-C₄)alkyl optionally substituted with one to three substituents independently selected from halo-, hydroxy, —CN, (C₁-C₄)alkoxy-, (C₂-C₄)alkenoxy, and (C₁-C₄)alkyl-SO₂—. Preferably, R³ is a nitrogen linked (C₁-C₁₀)heterocyclyl of formula (II), wherein R⁴ is hydrogen and R⁷ is (C₁-C₄)alkyl optionally substituted with one to three substituents independently selected from halo-, hydroxy, —CN, or (C₁-C₄)alkoxy-.

Still further, the invention provides compounds in which R³ is a nitrogen linked (C₁-C₁₀)heterocyclyl of formula (III), wherein R⁴ is hydrogen and R⁷ is (C₁-C₄)alkyl optionally substituted with one to three substituents independently selected from NH₂—, (C₁-C₄)alkyl)ₙ-N—, ((C₂-C₄)alkenyl), —N—, ((C₂-C₄)alkynyl)ₙ-N—, NH₂(C═O)—, (C₁-C₄)alkyl-(C═O)N—, ((C₁-C₄)alkyl)ₙ-N—(C═O)—, (C₂-C₄)alkenyl-(C═O)N—, ((C₂-C₄)alkenyl)ₙ-N—(C═O)—, (C₂-C₄)alkynyl-(C═O)N—, and ((C₂-C₄)alkynyl)ₙ-N—(C═O)—. Preferably, R³ is a nitrogen linked (C₁-C₁₀)heterocyclyl of formula (II), wherein R⁴ is hydrogen and R⁷ is (C₁-C₄)alkyl optionally substituted with one to three substituents independently selected from NH₂—, (C₁-C₄)alkyl)ₙ-N—, NH₂(C═O)—, (C₁-C₄)alkyl-(C═O)N—, and ((C₁-C₄)alkyl)ₙ-N—(C═O)—.

The methods of this invention may also utilize compounds in which R³ is a nitrogen linked (C₁-C₁₀)heterocyclyl of formula (III), wherein R⁴ is hydrogen and R⁷ is (C₁-C₄)alkyl optionally substituted with one to three substituents independently selected from (C₁-C₄)alkyl-(C═O)—, (C₂-C₄)alkenyl-(C═O)—, (C₂-C₄)alkynyl-(C═O)—, (C₃-C₁₀)cycloalkyl-(C═O)—, ((C₁-C₁₀)heterocyclyl-(C═O)—, (C₆-C₁₀)aryl-(C═O), and (C₁-C₁₀)heteroaryl-(C═O), and preferably, R³ is a nitrogen linked (C₁-C₁₀)heterocyclyl of formula (II), wherein R⁴ is hydrogen and R⁷ is (C₁-C₄)alkyl optionally substituted with one to three substituents independently selected from (C₁-C₄)alkyl-(C═O)—, (C₃-C₁₀)cycloalkyl-(C═O)—, ((C₁-C₁₀)heterocyclyl-(C═O)—, (C₆-C₁₀)aryl-(C═O), and (C₁-C₁₀)heteroaryl-(C═O).

Another embodiment of the invention includes the use in the present methods of compounds in which R³ is a nitrogen linked (C₁-C₁₀)heterocyclyl of formula (III), wherein R⁴ is hydrogen and R⁷ is (C₁-C₄)alkyl optionally substituted with one to three substituents independently selected from (C₁-C₄)alkyl-(C═O)O—, (C₂-C₄)alkenyl-(C═O)O—, (C₂-C₄)alkynyl-(C═O)O—, (C₁-C₄)alkyl-O(C═O)—, (C₂-C₄)alkenyl-O—(C═O)—, and (C₂-C₄)alkynyl-O—(C═O)—. Preferably, R³ is a nitrogen linked (C₁-C₁₀)heterocyclyl of formula (II), wherein R⁴ is hydrogen and R⁷ is (C₁-C₄)alkyl optionally substituted with one to three substituents independently selected from (C₁-C₄)alkyl-(C═O)O— and (C₁-C₄)alkyl-O(C═O)—.

Furthermore, the invention provides compounds in which R³ is a nitrogen linked (C₁-C₁₀)heterocyclyl of formula (III), wherein R⁴ is hydrogen and R⁷ is (C₁-C₄)alkyl optionally substituted with one to three substituents independently selected from (C₃-C₁₀)cycloalkyl-, (C₆-C₁₀)aryl-, (C₁-C₁₀)heterocyclyl-, and (C₁-C₁₀)heteroaryl-.

The methods of treatment and pharmaceutical compositions of the present invention may also utilize compounds of formula (I) wherein R³ is a nitrogen linked (C₁-C₁₀)heterocyclyl of formula (IV): 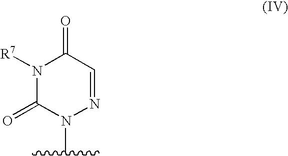
and R⁷ is selected from the group consisting of: 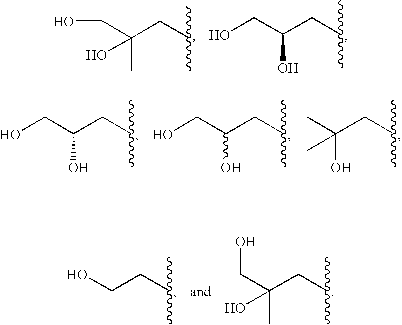
Also provided herein is the use of compounds of formula (I) wherein R³ is a nitrogen linked (C₁-C₁₀)heterocyclyl of formula (IV), and R⁷ is

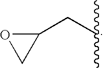

The present invention also contemplates use of compounds of formula (I) wherein R³ is a nitrogen linked (C₁-C₁₀)heterocyclyl of formula (IV), and R⁷ is selected from the group consisting of:

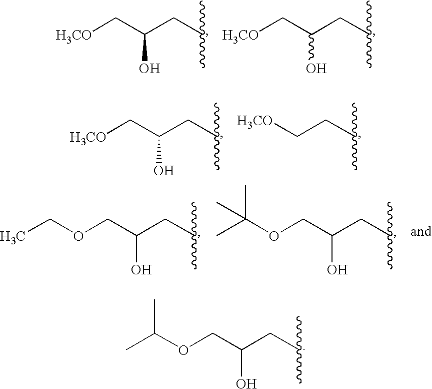

Also provided is the use of compounds of formula (I) wherein R³ is a nitrogen linked (C₁-C₁₀)heterocyclyl of formula (IV), and R⁷ is

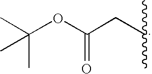

Further provided for is the use of compounds of formula (I) wherein R³ is a nitrogen linked (C₁-C₁₀)heterocyclyl of formula (IV), and R⁷ is selected from:

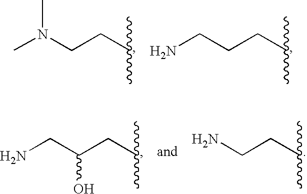

Finally, the invention further provides use in the methods herein of compounds of formula (I) wherein R³ is a nitrogen linked (C₁-C₁₀)heterocyclyl of formula (IV), and R⁷ is selected from:

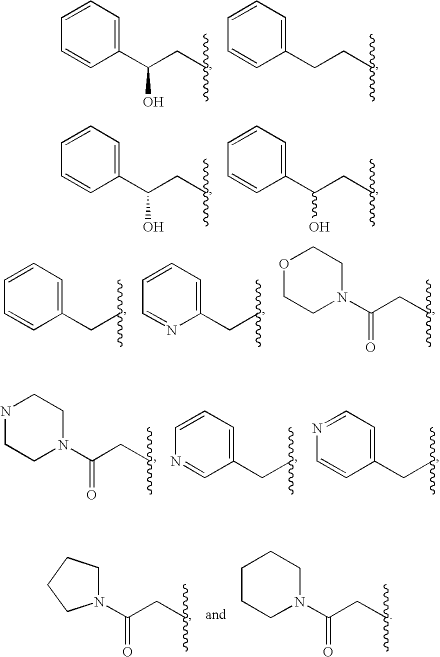

The present methods also provide compounds of formula (I) wherein R³ is a nitrogen linked (C₁-C₁₀)heterocyclyl of formula (IV), wherein R⁷ is selected from the group consisting of: 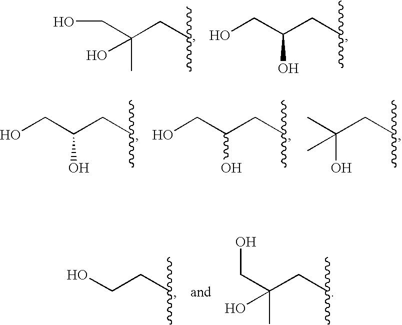
and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present invention also includes the use of compounds of formula (I) wherein R³ is a nitrogen linked (C₁-C₁₀)heterocyclyl of formula (IV), R⁷ is selected from the group consisting of: 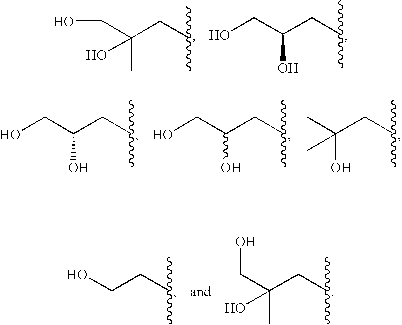
and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided is the use of compounds of formula (I) wherein R³ is a nitrogen linked (C₁-C₁₀)heterocyclyl of formula (IV),
R⁷ is 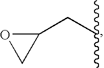
and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided is the use of compounds of formula (I) wherein R³ is a nitrogen linked (C₁-C₁₀)heterocyclyl of formula (IV), R⁷ is 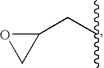
and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present invention also contemplates the use in the methods herein of compounds of formula (I) wherein R³ is a nitrogen linked (C₁-C₁₀)heterocyclyl of formula (IV), wherein R⁷ is selected from the group consisting of: 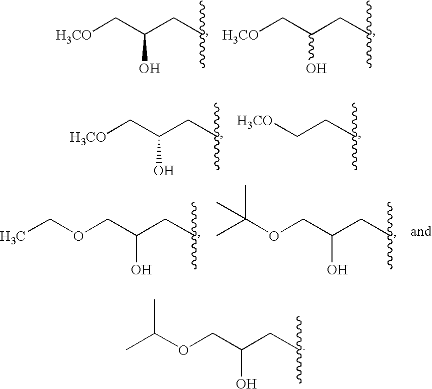
and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present invention further contemplates use of compounds of formula (I) wherein R³ is a nitrogen linked (C₁-C₁₀)heterocyclyl of formula (IV), R⁷ is selected from the group consisting of: 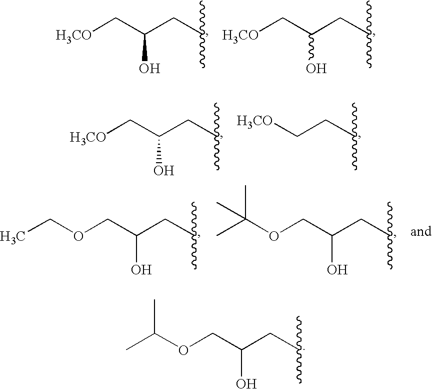
and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided for is the use of compounds of formula (I) wherein R³ is a nitrogen linked (C₁-C₁₀)heterocyclyl of formula (IV), R⁷ is 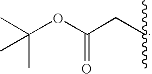
and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Additionally provided is the use in the methods herein of compounds of formula (I) wherein R³ is a nitrogen linked (C₁-C₁₀)heterocyclyl of formula (IV), R⁷ is 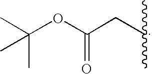
and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided are compounds of formula (I) wherein R³ is a nitrogen linked (C₁-C₁₀)heterocyclyl of formula (IV), R⁷ is selected from: 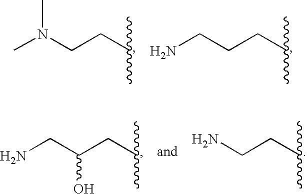
and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided herein is the use of compounds of formula (I) wherein R³ is a nitrogen linked (C₁-C₁₀)heterocyclyl of formula (IV), R⁷ is selected from: 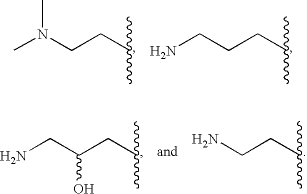
and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present methods further provides use of compounds of formula (I) wherein R³ is a nitrogen linked (C₁-C₁₀)heterocyclyl of formula (IV), R⁷ is selected from: 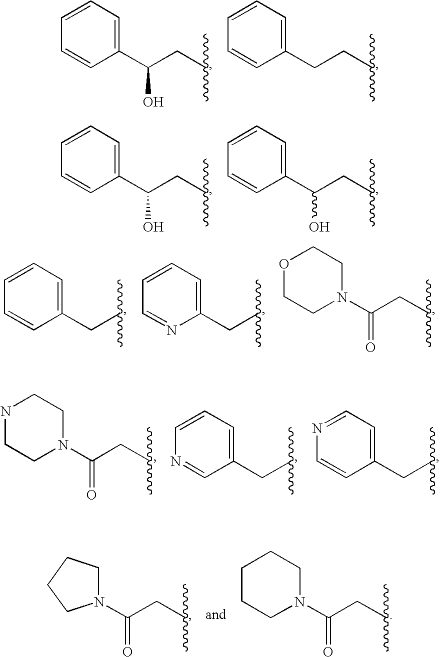
and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Finally, the methods of this invention further provide the use of compounds of formula (I) wherein R³ is a nitrogen linked (C₁-C₁₀)heterocyclyl of formula (IV), R⁷ is selected from: 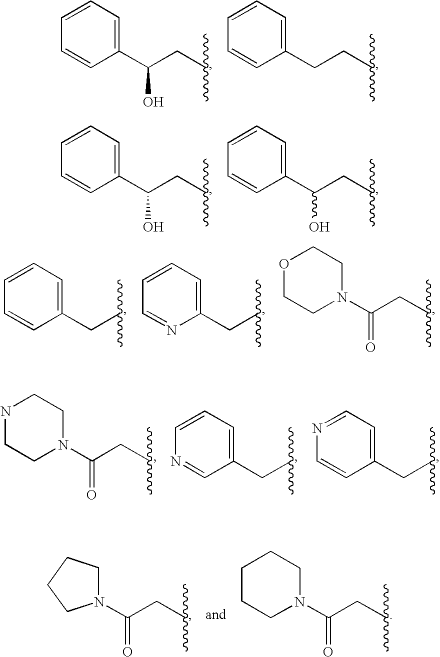
and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present invention also provides for use of compounds of formula (I) wherein R³ is a nitrogen linked (C₁-C₁₀)heterocyclyl of formula (IV), R⁷ is selected from the group consisting of: 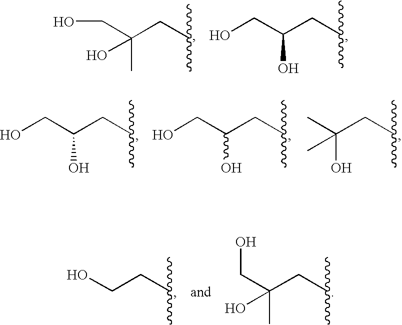
R² is chloro, methyl or ethyl; and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present methods may also utilize P2X₇ receptor inhibiting compounds of formula (I) wherein R³ is a nitrogen linked (C₁-C₁₀)heterocyclyl of formula (IV), R⁷ is selected from the group consisting of: 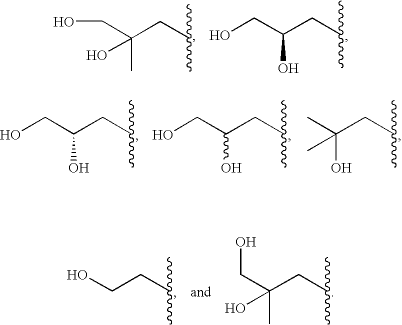
R² is chloro, methyl or ethyl; and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided herein is the use of compounds of formula (I) wherein R³ is a nitrogen linked (C₁-C₁₀)heterocyclyl of formula (IV), R⁷ is 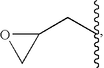
R² is chloro, methyl or ethyl; and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided is the use in the present methods of compounds of formula (I) wherein R³ is a nitrogen linked (C₁-C₁₀)heterocyclyl of formula (IV), R⁷ is 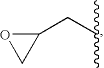
R² is chloro, methyl or ethyl; and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present invention also contemplates compounds of formula (I) wherein R³ is a nitrogen linked (C₁-C₁₀)heterocyclyl of formula (IV), R⁷ is selected from the group consisting of: 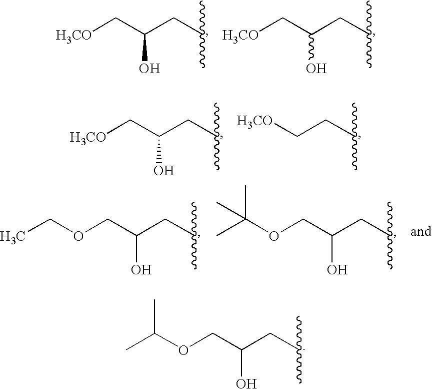
R² is chloro, methyl or ethyl; and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The present invention also contemplates the use of compounds of formula (I) wherein R³ is a nitrogen linked (C₁-C₁₀)heterocyclyl of formula (IV), R⁷ is selected from the group consisting of: 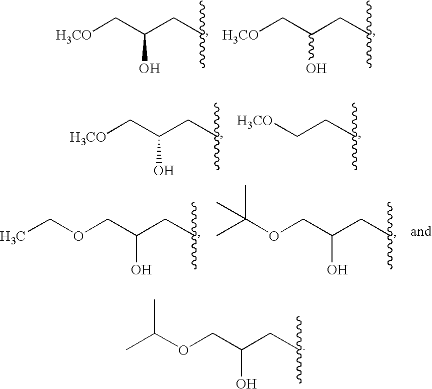
R² is chloro, methyl or ethyl; and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided is the use in the present methods of compounds of formula (I) wherein R³ is a nitrogen linked (C₁-C₁₀)heterocyclyl of formula (IV), R⁷ is 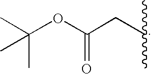
R² is chloro, methyl or ethyl; and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided are compounds of formula (I) wherein R³ is a nitrogen linked (C₁-C₁₀)heterocyclyl of formula (IV), R⁷ is 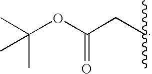
R² is chloro, methyl or ethyl; and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided is the use of compounds of formula (I) wherein R³ is a nitrogen linked (C₁-C₁₀)heterocyclyl of formula (IV), R⁷ is selected from: 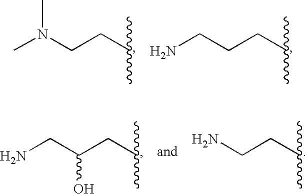
R² is chloro, methyl or ethyl; and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Also provided is the use in the present methods of compounds of formula (I) wherein R³ is a nitrogen linked (C₁-C₁₀)heterocyclyl of formula (IV), R⁷ is selected from: 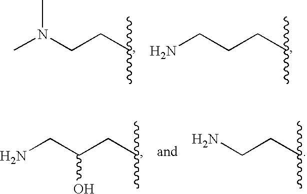
R² is chloro, methyl or ethyl; and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, CN, (C₁-C₆)alkyl, HO(C₁-C₆)alkyl, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

The invention further provides for the use of compounds of formula (I) wherein R³ is a nitrogen linked (C₁-C₁₀)heterocyclyl of formula (IV), R⁷ is selected from: 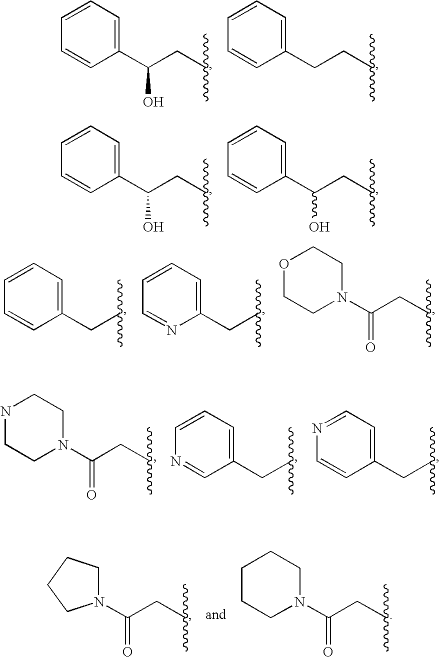
R² is chloro, methyl or ethyl; and R¹ is selected from the group consisting of (C₁-C₄)alkyl, optionally substituted by (C₃-C₁₀)cycloalkyl, wherein said (C₁-C₄)alkyl or (C₃-C₁₀)cycloalkyl are optionally substituted by one to three suitable moieties independently selected from the group consisting of hydroxy, halogen, —CN, (C₁-C₆)alkyl, HO(C₁-C₆)alkyl, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Finally, the methods of this invention further include the use of compounds of formula (I) wherein R³ is a nitrogen linked (C₁-C₁₀)heterocyclyl of formula (IV), R⁷ is selected from: 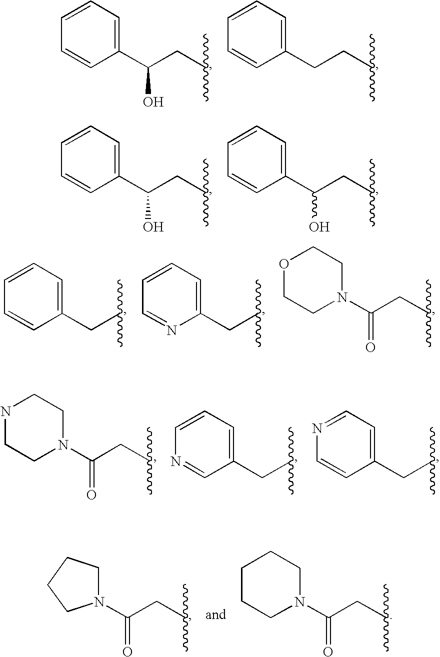
R² is chloro, methyl or ethyl; and R¹ is selected from the group consisting of (C₁-C₄)alkyl, optionally substituted by (C₆-C₁₀)aryl, wherein said (C₁-C₄)alkyl or (C₆-C₁₀)aryl are optionally substituted by one to three suitable moieties independently selected from the group consisting of hydroxy, halogen, —CN, (C₁-C₆)alkyl, HO(C₁-C₆)alkyl, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-.

Examples of other useful compounds of formula I are the following:
2-Chloro-N-(1-hydroxy-cycloheptylmethyl)-5-[4-(2-methanesulfonylamino-ethyl)-3,5-dioxo-4,5-dihydro-3H-[1,2,4]triazin-2-yl]-benzamide;
2-Chloro-5-[4-(2-formylamino-ethyl)-3,5-dioxo-4,5-dihydro-3H-[1,2,4]triazin-2-yl]-N-(1-hydroxy-cycloheptylmethyl)-benzamide;
5-[4-(1-Amino-cyclopropylmethyl)-3,5-dioxo-4,5-dihydro-3H-[1,2,4]triazin-2-yl]-2-chloro-N-(1-hydroxy-cycloheptylmethyl)-benzamide;
2-Chloro-N-(1-hydroxy-cycloheptylmethyl)-5-[4-(1-hydroxy-cyclopropylmethyl)-3,5-dioxo-4,5-dihydro-3H-[1,2,4]triazin-2-yl]-benzamide;
5-[4-(2-Amino-2-methyl-propyl)-3,5-dioxo-4,5-dihydro-3H-[1,2,4]triazin-2-yl]-2-chloro-N-(1-hydroxy-cycloheptylmethyl)-benzamide;
2-Chloro-5-[4-(3-difluoromethoxy-2-hydroxy-propyl)-3,5-dioxo-4,5-dihydro-3H-[1,2,4]triazin-2-yl]-N-(1-hydroxy-cycloheptylmethyl)-benzamide;
N-(1-Hydroxy-cycloheptylmethyl)-5-[4-(2-hydroxy-3-methoxy-propyl)-3,5-dioxo-4,5-dihydro-3H-[1,2,4]triazin-2-yl]-2-methyl-benzamide;
5-[4-(2-Hydroxy-3-methoxy-propyl)-3,5-dioxo-4,5-dihydro-3H-[1,2,4]triazin-2-yl]-N-(1-hydroxymethyl-cycloheptylmethyl)-2-methyl-benzamide;
N-(1-Hydroxy-cyclohexylmethyl)-5-[4-(2-hydroxy-3-methoxy-propyl)-3,5-dioxo-4,5-dihydro-3H-[1,2,4]triazin-2-yl]-2-methyl-benzamide;
1-({2-Chloro-5-[4-(2-hydroxy-3-methoxy-propyl)-3,5-dioxo-4,5-dihydro-3H-[1,2,4]triazin-2-yl]-benzoylamino}-methyl)-ycloheptanecarboxylicacid amide;
2-Chloro-N-[2-(2-chloro-phenyl)-ethyl]-5-[4-(2,3-dihydroxy-propyl)-3,5-dioxo-4,5-dihydro-3H-[1,2,4]triazin-2-yl]-benzamide;
2-Chloro-5-[4-(2,3-dihydroxy-propyl)-3,5-dioxo-4,5-dihydro-3H-[1,2,4]triazin-2-yl]-N-(1-hydroxymethyl-cycloheptylmethyl)-benzamide;
1-({2-Chloro-5-[4-(2,3-dihydroxy-propyl)-3,5-dioxo-4,5-dihydro-3H-[1,2,4]triazin-2-yl]-benzoylamino}-methyl)-cycloheptanecarboxylic acid amide;
2-Chloro-5-[4-(2,3-dihydroxy-propyl)-3,5-dioxo-4,5-dihydro-3H-[1,2,4]triazin-2-yl]-N-phenethyl-benzamide;
2-Chloro-5-[4-(2,3-dihydroxy-propyl)-3,5-dioxo-4,5-dihydro-3H-[1,2,4]triazin-2-yl]-N-(1-hydroxy-cyclohexylmethyl)-benzamide;
2-Chloro-5-[4-(2,3-dihydroxy-propyl)-3,5-dioxo-4,5-dihydro-3H-[1,2,4]triazin-2-yl]-N-(1-hydroxy-cyclopentylmethyl)-benzamide;
2-Chloro-5-[4-(2,3-dihydroxy-propyl)-3,5-dioxo-4,5-dihydro-3H-[1,2,4]triazin-2-yl]-N-(1-hydroxy-cyclobutylmethyl)-benzamide;
2-Chloro-N-(1-hydroxy-cycloheptylmethyl)-5-[4-(2-hydroxy-3-trifluoromethoxy-propyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-propyl)-3,5-dioxo-4,5-dihydro-3H-[1,2,4]triazin-2-yl]-benzamide;
2-Chloro-5-[4-(2-hydroxy-butyl)-3,5-dioxo-4,5-dihydro-3H-[1,2,4]triazin-2-yl]-N-(1-hydroxy-cycloheptylmethyl)-benzamide;
5-[4-(2-Amino-propyl)-3,5-dioxo-4,5-dihydro-3H-[1,2,4]triazin-2-yl]-2-chloro-N-(1-hydroxy-cycloheptylmethyl)-benzamide;
2-Chloro-5-[3,5-dioxo-4-(2-oxo-propyl)-4,5-dihydro-3H-[1,2,4]triazin-2-yl]-N-(1-hydroxy-cycloheptylmethyl)-benzamide;
2-Chloro-5-[3,5-dioxo-4-(2-oxo-ethyl)-4,5-dihydro-3H-[1,2,4]triazin-2-yl]-N-(1-hydroxy-cycloheptylmethyl)-benzamide;
2-Chloro-5-[3,5-dioxo-4-(2-trifluoromethoxy-ethyl)-4,5-dihydro-3H-[1,2,4]triazin-2-yl]-N-(1-hydroxy-cycloheptylmethyl)-benzamide;
2-Chloro-5-[4-(1-hydroxy-cyclobutylmethyl)-3,5-dioxo-4,5-dihydro-3H-[1,2,4]triazin-2-yl]-N-(1-hydroxy-cycloheptylmethyl)-benzamide;
2-Chloro-N-(1-hydroxy-cycloheptylmethyl)-5-[4-(2-hydroxy-2-pyridin-4-yl-ethyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-2-pyridin-3-yl-ethyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-2-pyridin-2-yl-ethyl)-3,5-dioxo-4,5-dihydro-3H-[1,2,4]triazin-2-yl]-benzamide;
2-Chloro-N-(1-hydroxy-cycloheptylmethyl)-5-[4-(4-hydroxy-tetrahydro-pyran-4-ylmethyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-2-thiophen-2-yl-ethyl)-3,5-dioxo-4,5-dihydro-3H-[1,2,4]triazin-2-yl]-benzamide;
2-Chloro-5-[4-(2-furan-2-yl-2-hydroxy-ethyl)-3,5-dioxo-4,5-dihydro-3H-[1,2,4]triazin-2-yl]-N-(1-hydroxy-cycloheptylmethyl)-benzamide;
5-(4-Carbamoylmethyl-3,5-dioxo-4,5-dihydro-3H-[1,2,4]triazin-2-yl)-2-chloro-N-[2-(2-chloro-phenyl)-ethyl]-benzamide;
2-Chloro-N-[2-(2-chloro-phenyl)-ethyl]-5-[4-(2-methoxy-ethyl)-3,5-dioxo-4,5-dihydro-3H-[1,2,4]triazin-2-yl]-benzamide;
5-[4-(2-Carbamoyl-ethyl)-3,5-dioxo-4,5-dihydro-3H-[1,2,4]triazin-2-yl]-2-chloro-N-[2-(2-chloro-phenyl)-ethyl]-benzamide;
2-Chloro-N-[2-(2-chloro-phenyl)-ethyl]-5-[4-(2-hydroxy-ethyl)-3,5-dioxo-4,5-dihydro-3H-[1,2,4]triazin-2-yl]-benzamide;
2-Chloro-N-[2-(2-chloro-phenyl)-ethyl]-5-[4-(3-hydroxy-propyl)-3,5-dioxo-4,5-dihydro-3H-[1,2,4]triazin-2-yl]-benzamide;
5-(4-Carbamoylmethyl-3,5-dioxo-4,5-dihydro-3H-[1,2,4]triazin-2-yl)-2-chloro-N-(1-hydroxy-cycloheptylmethyl)-benzamide;
2-Chloro-N-(1-hydroxy-cycloheptylmethyl)-5-[4-(2-hydroxy-ethyl)-3,5-dioxo-4,5-dihydro-3H-[1,2,4]triazin-2-yl]-benzamide;
2-Chloro-N-(1-hydroxy-cycloheptylmethyl)-5-[4-(2-methoxy-ethyl)-3,5-dioxo-4,5-dihydro-3H-[1,2,4]triazin-2-yl]-benzamide;
2-Chloro-5-[4-(2-dimethylamino-ethyl)-3,5-dioxo-4,5-dihydro-3H-[1,2,4]triazin-2-yl]-N-(1-hydroxy-cycloheptylmethyl)-benzamide;
2-Chloro-5-(4-cyanomethyl-3,5-dioxo-4,5-dihydro-3H-[1,2,4]triazin-2-yl)-N-(1-hydroxy-cycloheptylmethyl)-benzamide;
2-Chloro-N-(1-hydroxy-cycloheptylmethyl)-5-[4-(3-hydroxy-propyl)-3,5-dioxo-4,5-dihydro-3H-[1,2,4]triazin-2-yl]-benzamide;
5-[4-(2-Amino-ethyl)-3,5-dioxo-4,5-dihydro-3H-[1,2,4]triazin-2-yl]-2-chloro-N-(1-hydroxy-cycloheptylmethyl)-benzamide;
2-Chloro-N-(1-hydroxy-cycloheptylmethyl)-5-(4-oxiranylmethyl-3,5-dioxo-4,5-dihydro-3H-[1,2,4]triazin-2-yl)-benzamide;
5-[4-(2-Acetylamino-ethyl)-3,5-dioxo-4,5-dihydro-3H-[1,2,4]triazin-2-yl]-2-chloro-N-(1-hydroxy-cycloheptylmethyl)-benzamide;
2-Chloro-5-[4-(2,3-dihydroxy-propyl)-3,5-dioxo-4,5-dihydro-3H-[1,2,4]triazin-2-yl]-N-(1-hydroxy-cycloheptylmethyl)-benzamide;
2-Chloro-5-(3,5-dioxo-4-phenethyl-4,5-dihydro-3H-[1,2,4]triazin-2-yl)-N-(1-hydroxy-cycloheptylmethyl)-benzamide;
5-(4-Benzyl-3,5-dioxo-4,5-dihydro-3H-[1,2,4]triazin-2-yl)-2-chloro-N-(1-hydroxy-cycloheptylmethyl)-benzamide;
2-Chloro-5-[4-(2-cyano-ethyl)-3,5-dioxo-4,5-dihydro-3H-[1,2,4]triazin-2-yl]-N-(1-hydroxy-cycloheptylmethyl)-benzamide;
2-Chloro-5-(3,5-dioxo-4-pyridin-2-ylmethyl-4,5-dihydro-3H-[1,2,4]triazin-2-yl)-N-(1-hydroxy-cycloheptylmethyl)-benzamide;
2-Chloro-5-(3,5-dioxo-4-pyridin-3-ylmethyl-4,5-dihydro-3H-[1,2,4]triazin-2-yl)-N-(1-hydroxy-cycloheptylmethyl)-benzamide;
2-Chloro-5-(3,5-dioxo-4-pyridin-4-ylmethyl-4,5-dihydro-3H-[1,2,4]triazin-2-yl)-N-(1-hydroxy-cycloheptylmethyl)-benzamide;
5-[4-(2-Carbamoyl-ethyl)-3,5-dioxo-4,5-dihydro-3H-[1,2,4]triazin-2-yl]-2-chloro-N-(1-hydroxy-cycloheptylmethyl)-benzamide;
N-(1-Hydroxy-cycloheptylmethyl)-5-[4-(2-hydroxy-ethyl)-3,5-dioxo-4,5-dihydro-3H-[1,2,4]triazin-2-yl]-2-methyl-benzamide;
5-[4-(2,3-Dihydroxy-propyl)-3,5-dioxo-4,5-dihydro-3H-[1,2,4]triazin-2-yl]-N-(1-hydroxy-cycloheptylmethyl)-2-methyl-benzamide;
5-[4-(2-Carbamoyl-ethyl)-3,5-dioxo-4,5-dihydro-3H-[1,2,4]triazin-2-yl]-N-(1-hydroxy-cycloheptylmethyl)-2-methyl-benzamide;
5-[4-(3-Amino-propyl)-3,5-dioxo-4,5-dihydro-3H-[1,2,4]triazin-2-yl]-2-chloro-N-(1-hydroxy-cycloheptylmethyl)-benzamide;
2-Chloro-5-[4-(2,3-dihydroxy-propyl)-3,5-dioxo-4,5-dihydro-3H-[1,2,4]triazin-2-yl]-N-(1-hydroxy-cycloheptylmethyl)-benzamide;
(2-{4-Chloro-3-[2-(2-chloro-phenyl)-ethylcarbamoyl]-phenyl}-3,5-dioxo-2,5-dihydro-3H-[1,2,4]triazin-4-yl)-acetic acid tert-butyl ester;
2-Chloro-5-[4-(2,3-dihydroxy-propyl)-3,5-dioxo-4,5-dihydro-3H-[1,2,4]triazin-2-yl]-N-(1-hydroxy-cycloheptylmethyl)-benzamide;
5-(4-Carbamoylmethyl-3,5-dioxo-4,5-dihydro-3H-[1,2,4]triazin-2-yl)-2-chloro-N-(1-cyano-cycloheptylmethyl)-benzamide;
N-Adamantan-1-ylmethyl-5-(4-carbamoylmethyl-3,5-dioxo-4,5-dihydro-3H-[1,2,4]triazin-2-yl)-2-chloro-benzamide;
5-(4-Carbamoylmethyl-3,5-dioxo-4,5-dihydro-3H-[1,2,4]triazin-2-yl)-2-chloro-N-(4,4-difluoro-1-phenyl-cyclohexylmethyl)-benzamide;
5-(4-Carbamoylmethyl-3,5-dioxo-4,5-dihydro-3H-[1,2,4]triazin-2-yl)-2-chloro-N-(1-p-tolyl-cyclohexylmethyl)-benzamide;
5-(4-Carbamoylmethyl-3,5-dioxo-4,5-dihydro-3H-[1,2,4]triazin-2-yl)-2-chloro-N-(1-hydroxy-cyclohexylmethyl)-benzamide;
2-Chloro-N-(1-hydroxy-cyclohexylmethyl)-5-[4-(2-hydroxy-ethyl)-3,5-dioxo-4,5-dihydro-3H-[1,2,4]triazin-2-yl]-benzamide;
2-Chloro-N-(1-hydroxy-3,3-dimethyl-cyclohexylmethyl)-5-[4-(2-hydroxy-ethyl)-3,5-dioxo-4,5-dihydro-3H-[1,2,4]triazin-2-yl]-benzamide;
2-Chloro-N-(1-hydroxy-cyclooctylmethyl)-5-[4-(2-hydroxy-ethyl)-3,5-dioxo-4,5-dihydro-3H-[1,2,4]triazin-2-yl]-benzamide;
2-Chloro-5-[4-(2,3-dihydroxy-propyl)-3,5-dioxo-4,5-dihydro-3H-[1,2,4]triazin-2-yl]-N-(1-hydroxy-cyclohexylmethyl)-benzamide;
5-[4-(2,3-Dihydroxy-propyl)-3,5-dioxo-4,5-dihydro-3H-[1,2,4]triazin-2-yl]-N-(1-hydroxy-cycloheptylmethyl)-2-methyl-benzamide;
5-[4-(2,3-Dihydroxy-propyl)-3,5-dioxo-4,5-dihydro-3H-[1,2,4]triazin-2-yl]-N-(1-hydroxy-cycloheptylmethyl)-2-methyl-benzamide;
5-(4-Carbamoylmethyl-3,5-dioxo-4,5-dihydro-3H-[1,2,4]triazin-2-yl)-2-chloro-N-(1-hydroxy-cyclooctylmethyl)-benzamide;
2-Chloro-N-(2-hydroxy-cycloheptylmethyl)-5-[4-(2-hydroxy-ethyl)-3,5-dioxo-4,5-dihydro-3H-[1,2,4]triazin-2-yl]-benzamide;
5-[4-(3-Amino-2-hydroxy-propyl)-3,5-dioxo-4,5-dihydro-3H-[1,2,4]triazin-2-yl]-2-chloro-N-(1-hydroxy-cycloheptylmethyl)-benzamide;
2-Chloro-N-(1-hydroxy-cycloheptylmethyl)-5-(4-methylcarbamoylmethyl-3,5-dioxo-4,5-dihydro-3H-[1,2,4]triazin-2-yl)-benzamide;
2-Chloro-N-[2-(2-chloro-phenyl)-ethyl]-5-(4-dimethylcarbamoylmethyl-3,5-dioxo-4,5-dihydro-3H-[1,2,4]triazin-2-yl)-benzamide;
2-Chloro-N-[2-(2-chloro-phenyl)-ethyl]-5-[4-(2-morpholin-4-yl-2-oxo-ethyl)-3,5-dioxo-4,5-dihydro-3H-[1,2,4]triazin-2-yl]-benzamide;
2-Chloro-N-[2-(2-chloro-phenyl)-ethyl]-5-[3,5-dioxo-4-(2-oxo-2-pyrrolidin-1-yl-ethyl)-4,5-dihydro-3H-[1,2,4]triazin-2-yl]-benzamide;
2-Chloro-N-[2-(2-chloro-phenyl)-ethyl]-5-[4-(2-methylcarbamoyl-ethyl)-3,5-dioxo-4,5-dihydro-3H-[1,2,4]triazin-2-yl]-benzamide;
2-Chloro-N-[2-(2-chloro-phenyl)-ethyl]-5-[4-(2-dimethylcarbamoyl-ethyl)-3,5-dioxo-4,5-dihydro-3H-[1,2,4]triazin-2-yl]-benzamide;
2-Chloro-N-[2-(2-chloro-phenyl)-ethyl]-5-[3,5-dioxo-4-(2-oxo-2-piperazin-1-yl-ethyl)-4,5-dihydro-3H-[1,2,4]triazin-2-yl]-benzamide;
2-Chloro-5-(4-dimethylcarbamoylmethyl-3,5-dioxo-4,5-dihydro-3H-[1,2,4]triazin-2-yl)-N-(1-hydroxy-cycloheptylmethyl)-benzamide;
2-Chloro-5-(4-ethylcarbamoylmethyl-3,5-dioxo-4,5-dihydro-3H-[1,2,4]triazin-2-yl)-N-(1-hydroxy-cycloheptylmethyl)-benzamide;
2-Chloro-5-[3,5-dioxo-4-(2-oxo-2-piperidin-1-yl-ethyl)-4,5-dihydro-3H-[1,2,4]triazin-2-yl]-N-(1-hydroxy-cycloheptylmethyl)-benzamide;
2-Chloro-N-(1-hydroxy-cycloheptylmethyl)-5-[4-(2-morpholin-4-yl-2-oxo-ethyl)-3,5-dioxo-4,5-dihydro-3H-[1,2,4]triazin-2-yl]-benzamide;
2-Chloro-N-(1-hydroxy-cycloheptylmethyl)-5-[4-(isopropylcarbamoyl-methyl)-3,5-dioxo-4,5-dihydro-3H-[1,2,4]triazin-2-yl]-benzamide;
2-Chloro-5-[3,5-dioxo-4-(2-oxo-2-pyrrolidin-1-yl-ethyl)-4,5-dihydro-3H-[1,2,4]triazin-2-yl]-N-(1-hydroxy-cycloheptylmethyl)-benzamide;
2-Chloro-5-{4-[(cyclopropylmethyl-carbamoyl)-methyl]-3,5-dioxo-4,5-dihydro-3H-[1,2,4]triazin-2-yl}-N-(1-hydroxy-cycloheptylmethyl)-benzamide;
5-(4-Dimethylcarbamoylmethyl-3,5-dioxo-4,5-dihydro-3H-[1,2,4]triazin-2-yl)-N-(1-hydroxy-cycloheptylmethyl)-2-methyl-benzamide;
2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide;
2-Chloro-5-[4-(2,3-dihydroxy-2-methyl-propyl)-3,5-dioxo-4,5-dihydro-3H-[1,2,4]triazin-2-yl]-N-(1-hydroxy-cycloheptylmethyl)-benzamide;
2-Chloro-N-(1-hydroxy-cycloheptylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cyclohexylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cyclooctylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cyclooctylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cyclohexylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cyclooctylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cyclopentylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cyclopentylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cyclopentylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cyclobutylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cyclobutylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cyclopentylmethyl)-5-[4-(2-hydroxy-3-methoxy-propyl)-3,5-dioxo-4,5-dihydro-3H-[1,2,4]triazin-2-yl]-benzamide;
2-Chloro-5-[4-(2-hydroxy-3-methoxy-propyl)-3,5-dioxo-4,5-dihydro-3H-[1,2,4]triazin-2-yl]-N-(1-hydroxymethyl-cycloheptylmethyl)-benzamide;
2-Chloro-N-(1-hydroxymethyl-cycloheptylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-2-phenyl-ethyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-2-phenyl-ethyl)-3,5-dioxo-4,5-dihydro-3H-[1,2,4]triazin-2-yl]-benzamide;
2-Chloro-5-[4-(3-ethoxy-2-hydroxy-propyl)-3,5-dioxo-4,5-dihydro-3H-[1,2,4]triazin-2-yl]-N-(1-hydroxy-cycloheptylmethyl)-benzamide;
2-Chloro-N-(1-hydroxy-cycloheptylmethyl)-5-[4-(2-hydroxy-3-isopropoxy-propyl)-3,5-dioxo-4,5-dihydro-3H-[1,2,4]triazin-2-yl]-benzamide;
5-[4-(3-tert-Butoxy-2-hydroxy-propyl)-3,5-dioxo-4,5-dihydro-3H-[1,2,4]triazin-2-yl]-2-chloro-N-(1-hydroxy-cycloheptylmethyl)-benzamide;
2-Chloro-N-[2-(2-chloro-phenyl)-ethyl]-5-[4-(2-hydroxy-3-methoxy-propyl)-3,5-dioxo-4,5-dihydro-3H-[1,2,4]triazin-2-yl]-benzamide;
2-Chloro-5-[3,5-dioxo-4-(3,3,3-trifluoro-2-hydroxy-propyl)-4,5-dihydro-3H-[1,2,4]triazin-2-yl]-N-(1-hydroxy-cycloheptylmethyl)-benzamide;
2-Chloro-N-(1-hydroxy-cycloheptylmethyl)-5-[4-(2-hydroxy-3,3-dimethyl-butyl)-3,5-dioxo-4,5-dihydro-3H-[1,2,4]triazin-2-yl]-benzamide;
3-(2-{4-Chloro-3-[(1-hydroxy-cycloheptylmethyl)-carbamoyl]-phenyl}-3,5-dioxo-2,5-dihydro-3H-[1,2,4]triazin-4-yl)-2-hydroxy-2-methyl-propionic acid methyl ester;
2-Chloro-N-(1-hydroxy-cycloheptylmethyl)-5-[4-(2-hydroxy-3-morpholin-4-yl-propyl)-3,5-dioxo-4,5-dihydro-3H-[1,2,4]triazin-2-yl]-benzamide;
5-[4-(3-Benzyloxy-2-hydroxy-propyl)-3,5-dioxo-4,5-dihydro-3H-[1,2,4]triazin-2-yl]-2-chloro-N-(1-hydroxy-cycloheptylmethyl)-benzamide;
2-Chloro-N-[2-(2-chloro-phenyl)-ethyl]-5-[4-(2-hydroxy-2-methyl-propyl)-3,5-dioxo-4,5-dihydro-3H-[1,2,4]triazin-2-yl]-benzamide;
2-Chloro-N-[2-(2-chloro-phenyl)-ethyl]-5-[4-(2-hydroxy-2-phenyl-ethyl)-3,5-dioxo-4,5-dihydro-3H-[1,2,4]triazin-2-yl]-benzamide;
2-Chloro-N-(2-hydroxy-cycloheptylmethyl)-5-[4-(2-hydroxy-2-methyl-propyl)-3,5-dioxo-4,5-dihydro-3H-[1,2,4]triazin-2-yl]-benzamide;
2-Chloro-N-(2-hydroxy-cycloheptylmethyl)-5-[4-(2-hydroxy-2-phenyl-ethyl)-3,5-dioxo-4,5-dihydro-3H-[1,2,4]triazin-2-yl]-benzamide;
2-Chloro-N-(2-hydroxy-cycloheptylmethyl)-5-[4-(2-hydroxy-3-methoxy-propyl)-3,5-dioxo-4,5-dihydro-3H-[1,2,4]triazin-2-yl]-benzamide;
2-Chloro-5-[4-(2-hydroxy-3-methoxy-propyl)-3,5-dioxo-4,5-dihydro-3H-[1,2,4]triazin-2-yl]-N-(2-hydroxy-2-phenyl-ethyl)-benzamide;
2-Chloro-5-[4-(2-hydroxy-3-methoxy-propyl)-3,5-dioxo-4,5-dihydro-3H-[1,2,4]triazin-2-yl]-N-(2-hydroxy-2-phenyl-ethyl)-benzamide;
2-Chloro-5-[4-(2-hydroxy-2-methyl-propyl)-3,5-dioxo-4,5-dihydro-3H-[1,2,4]triazin-2-yl]-N-(2-hydroxy-2-phenyl-ethyl)-benzamide;
2-Chloro-5-[4-(2-hydroxy-3-methoxy-propyl)-3,5-dioxo-4,5-dihydro-3H-[1,2,4]triazin-2-yl]-N-phenethyl-benzamide; and
2-Chloro-5-[4-(2-hydroxy-2-methyl-propyl)-3,5-dioxo-4,5-dihydro-3H-[1,2,4]triazin-2-yl]-N-(2-hydroxy-2-phenyl-ethyl)-benzamide.

The present invention also includes the use in the present methods of treatment the following preferred compounds:
2-Chloro-N-(1-hydroxy-cyclohexylmethyl)-5-[4-(2-hydroxy-3-methoxy-propyl)-3,5-dioxo-4,5-dihydro-3H-[1,2,4]triazin-2-yl]-benzamide;
2-Chloro-5-[4-(2,3-dihydroxy-propyl)-3,5-dioxo-4,5-dihydro-3H-[1,2,4]triazin-2-yl]-N-(1-hydroxy-cyclohexylmethyl)-benzamide;
2-Chloro-N-(1-hydroxy-cycloheptylmethyl)-5-[4-(2-hydroxy-ethyl)-3,5-dioxo-4,5-dihydro-3H-[1,2,4]triazin-2-yl]-benzamide;
2-Chloro-5-[4-(2,3-dihydroxy-propyl)-3,5-dioxo-4,5-dihydro-3H-[1,2,4]triazin-2-yl]-N-(1-hydroxy-cycloheptylmethyl)-benzamide;
2-Chloro-5-(4-cyanomethyl-3,5-dioxo-4,5-dihydro-3H-[1,2,4]triazin-2-yl)-N-(1-hydroxy-cycloheptylmethyl)-benzamide;
2-Chloro-5-[4-(2-hydroxy-3-methoxy-propyl)-3,5-dioxo-4,5-dihydro-3H-[1,2,4]triazin-2-yl]-N-(1-hydroxymethyl-cycloheptylmethyl)-benzamide;
2-Chloro-5-[4-(2-cyano-ethyl)-3,5-dioxo-4,5-dihydro-3H-[1,2,4]triazin-2-yl]-N-(1-hydroxy-cycloheptylmethyl)-benzamide;
N-(1-Hydroxy-cycloheptylmethyl)-5-[4-(2-hydroxy-ethyl)-3,5-dioxo-4,5-dihydro-3H-[1,2,4]triazin-2-yl]-2-methyl-benzamide;
2-Chloro-5-[4-(2,3-dihydroxy-propyl)-3,5-dioxo-4,5-dihydro-3H-[1,2,4]triazin-2-yl]-N-(1-hydroxy-cyclohexylmethyl)-benzamide;
2-Chloro-N-(1-hydroxy-cycloheptylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cyclooctylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-2-phenyl-ethyl)-3,5-dioxo-4,5-dihydro-3H-[1,2,4]triazin-2-yl]-benzamide;
2-Chloro-5-[3,5-dioxo-4-(3,3,3-trifluoro-2-hydroxy-propyl)-4,5-dihydro-3H-[1,2,4]triazin-2-yl]-N-(1-hydroxy-cycloheptylmethyl)-benzamide;
2-Chloro-5-[4-(2-hydroxy-3-methoxy-propyl)-3,5-dioxo-4,5-dihydro-3H-[1,2,4]triazin-2-yl]-N-(2-hydroxy-2-phenyl-ethyl)-benzamide;
5-(4-Carbamoylmethyl-3,5-dioxo-4,5-dihydro-3H-[1,2,4]triazin-2-yl)-2-chloro-N-(1-hydroxy-cycloheptylmethyl)-benzamide;
2-Chloro-N-(1-hydroxy-cycloheptylmethyl)-5-[4-(2-methoxy-ethyl)-3,5-dioxo-4,5-dihydro-3H-[1,2,4]triazin-2-yl]-benzamide;
5-[4-(2,3-Dihydroxy-propyl)-3,5-dioxo-4,5-dihydro-3H-[1,2,4]triazin-2-yl]-N-(1-hydroxy-cycloheptylmethyl)-2-methyl-benzamide;
5-[4-(3-Amino-2-hydroxy-propyl)-3,5-dioxo-4,5-dihydro-3H-[1,2,4]triazin-2-yl]-2-chloro-N-(1-hydroxy-cycloheptylmethyl)-benzamide; and
2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide.

The present invention also includes the use of isotopically-labeled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labelled compounds of Formula I of this invention and prodrugs thereof can generally be prepared by carrying out the procedures disclosed in the Schemes and/or in the Examples and Preparations below, by substituting a readily available isotopically-labelled reagent for a non-isotopically-labelled reagent.

The combination of compounds of Formula I or a pharmaceutically acceptable salt thereof and a pharmaceutically effective amount of a pharmaceutical agent selected from the group of sulfasalazine, a statin, a glucocorticoid agent, an inhibitor of p38 kinase, an anti-IL-6-receptor antibody, anakinra (Kineret®), an IL-1 monoclonal antibody, an inhibitor of JAK3 protein tyrosine kinase, a macrophage colony stimulation factor (M-CSF) monoclonal antibody, a humanized anti-CD20 monoclonal antibody, as hereinbefore defined, can be used in the manufacture of a medicament for the prophylactic or therapeutic treatment of any disease state in a human, or other mammal, which is exacerbated or caused by excessive or unregulated cytokine production by such mammal's cells, such as but not limited to monocytes and/or macrophages.

The present invention also relates to a method for treating an IL-1 mediated condition. As defined herein, a “IL-1 mediated condition” includes but is not limited to a disease or disorder selected from the group consisting of arthritis (including psoriatic arthritis, Reiter's syndrome, rheumatoid arthritis, gout, traumatic arthritis, rubella arthritis, rheumatoid spondylitis, osteoarthritis, gouty arthritis and acute synovitis), inflammatory bowel disease, Crohn's disease, emphysema, acute respiratory distress syndrome, adult respiratory distress syndrome, asthma, bronchitis chronic obstructive pulmonary disease, chronic pulmonary inflammatory disease, silicosis, pulmonary sarcoidosis, allergic reactions, allergic contact hypersensitivity, eczema, contact dermatitis, psoriasis, sunburn, cancer, tissue ulceration, restenosis, periodontal disease, epidermolysis bullosa, osteoporosis, bone resorption disease, loosening of artificial joint implants, atherosclerosis, aortic aneurysm, congestive heart failure, myocardial infarction, stroke, cerebral ischemia, head trauma, neurotrauma, spinal cord injury, neuro-degenerative disorders, Alzheimer's disease, Parkinson's disease, migraine, depression, peripheral neuropathy, pain, cerebral amyloid angiopathy, nootropic or cognition enhancement, amyotrophic lateral sclerosis, multiple sclerosis, ocular angiogenesis, corneal injury, macular degeneration, corneal scarring, scleritis, abnormal wound healing, burns, autoimmune disorders, Huntington's disease, diabetes, AIDS, cachexia, sepsis, septic shock, endotoxic shock, conjunctivitis shock, gram negative sepsis, toxic shock syndrome, cerebral malaria, cardiac and renal reperfusion injury, thrombosis, glomerularonephritis, graft vs. host reaction, allograft rejection, organ transplant toxicity, ulcerative colitis, or muscle degeneration, in a mammal, including a human, comprising administering to said mammal an amount of a compound to formula I, effective in treating such a condition.

The present invention relates to a pharmaceutical composition which comprises an effective amount of a compound according to formula I and a pharmaceutically effective amount of a pharmaceutical agent selected from the group of sulfasalazine, a statin, a glucocorticoid agent, an inhibitor of p38 kinase, an anti-IL-6-receptor antibody, anakinra (Kineret®), an anti-IL-1 monoclonal antibody, an inhibitor of JAK3 protein tyrosine kinase, a macrophage colony stimulation factor (M-CSF) monoclonal antibody, anti-CD20 monoclonal antibody as hereinbefore defined and a pharmaceutically acceptable carrier.

Preferably, the combinations of pharmaceutically effective agents of the invention are useful for the treatment of rheumatoid arthritis, osteoarthritis, psoriasis, allergic dermatitis, asthma, chronic obstructive pulmonary disease (COPD), hyperresponsiveness of the airway, septic shock, glomerulonephritis, irritable bowel disease, Crohn's disease, ulcerative colitis, atherosclerosis, growth and metastases of malignant cells, myoblastic leukemia, diabetes, Alzheimer's disease, meningitis, osteoporosis, burn injury, ischemic heart disease, stroke and varicose veins.

The invention further provides a method of treating osteoarthritis which comprises administering a therapeutically effective amount of a compound of formula (I), or a pharmaceutically acceptable salt or solvate thereof, and a pharmaceutically effective amount of a pharmaceutical agent selected from the group of sulfasalazine, a statin, a glucocorticoid agent, an inhibitor of p38 kinase, an anti-IL-6-receptor antibody, anakinra (Kineret®), an anti-IL-1 monoclonal antibody, an inhibitor of JAK3 protein tyrosine kinase, a macrophage colony stimulation factor (M-CSF) monoclonal antibody, anti-CD20 monoclonal antibody as hereinbefore defined to a patient.

The invention further provides a method of effecting immunosuppression (e.g. in the treatment of rheumatoid arthritis, irritable bowel disease, atherosclerosis or psoriasis) which comprises administering a therapeutically effective amount of a compound of formula (I), or a pharmaceutically acceptable salt or solvate thereof, and a pharmaceutically effective amount of a pharmaceutical agent selected from the group of sulfasalazine, a statin, a glucocorticoid agent, an inhibitor of p38 kinase, an anti-IL-6-receptor antibody, anakinra (Kineret®), an anti-IL-1 monoclonal antibody, an inhibitor of JAK3 protein tyrosine kinase, a macrophage colony stimulation factor (M-CSF) monoclonal antibody, an anti-CD20 monoclonal antibody as hereinbefore defined to a patient.

The invention also provides a method of treating an obstructive airways disease (e.g. asthma or COPD) which comprises administering to a patient a therapeutically effective amount of a compound of formula (I), or a pharmaceutically acceptable salt or solvate thereof, and a pharmaceutically effective amount of a pharmaceutical agent selected from the group of sulfasalazine, a statin, a glucocorticoid agent, an inhibitor of p38 kinase, an anti-IL-6-receptor antibody, anakinra (Kineret®), an anti-IL-1 monoclonal antibody, an inhibitor of JAK3 protein tyrosine kinase, a macrophage colony stimulation factor (M-CSF) monoclonal antibody, an anti-CD20 monoclonal antibody as hereinbefore defined to a patient.

The term “treating”, as used herein, refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The present invention also provides a pharmaceutical composition comprising a compound of formula (I), or a pharmaceutically acceptable salt or solvate thereof, as hereinbefore defined in association with a pharmaceutically acceptable adjuvant, diluent or carrier.

A pharmaceutically or therapeutically effective amount or dose of a pharmaceutical agent useful in the methods, combinations and formulations of this invention will also be understood to be an amount sufficient to provide a preventative, inhibitory, ameliorating or diminishing effect on the maladies described herein, their symptoms or physiological origins.

For the above-mentioned therapeutic uses the dosage of the P2X₇ receptor-inhibiting compound administered will, of course, vary with the compound employed, the mode of administration, the treatment desired and the disorder indicated. The daily dosage of the compound of formula (I)/salt/solvate (active ingredient) may be in the range from 1 mg to 1 gram, preferably 1 mg to 250 mg, more preferably 10 mg to 100 mg.

The methods of the present invention also include the use of sustained release compositions containing a pharmaceutically or therapeutically useful amount of a P2X₇ receptor-inhibiting compound.

One of ordinary skill in the art will appreciate that the compounds of the invention are useful in treating a diverse array of diseases. One of ordinary skill in the art will also appreciate that when using the compounds of the invention in the treatment of a specific disease that the compounds of the invention may be combined with various existing therapeutic agents used for that disease.

As used herein, co-administered combinations of the above-mentioned pharmaceutically useful agents includes administering a compound of Formula I with a agent, as defined herein, using a dosing regimen that promotes the desired result. This can refer to simultaneous dosing, dosing at different times during a single day, or even dosing on different days. The compounds can be administered separately or can be combined into a single formulation, in cases wherein both may be formulated together in a solid form, liquid form, etc., by methods known in the art.

P2X₇ inhibiting compounds of the formula I may be prepared according to the following reaction schemes and discussion. Unless otherwise indicated R¹ through R⁷ in the reaction schemes and discussion that follows are as defined above.

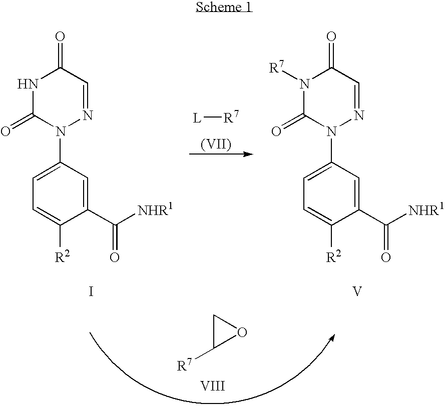

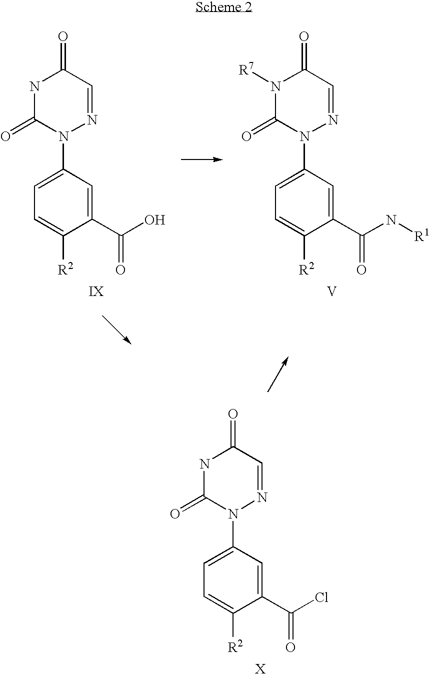

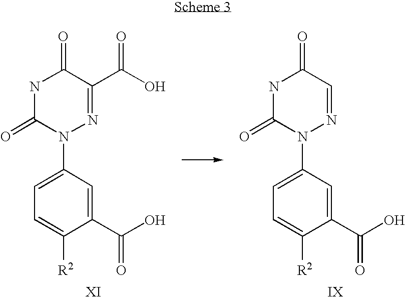

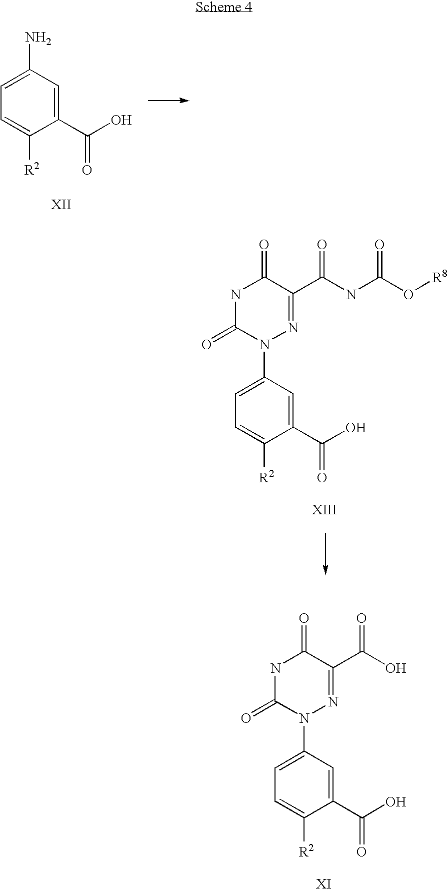

Scheme 1 refers to the preparation of compounds of the formula V. Compounds of the formula V can be prepared from compounds of formula I by reaction with a compound of the formula VII in the presence of base, wherein L is a suitable leaving group, such as chloro, bromo, iodo tosylate or mesylate. Suitable bases include, but are not limited to, triethylamine, polymer supported BEMP, cesium carbonate, potassium carbonate, and sodium hydride, where cesium carbonate is preferred. The aforesaid reaction can be performed at temperatures ranging from 0° C. to 100° C. in the presence of a polar solvent including but not limited to dimethylsulfoxide, dimethylformamide, equal amounts of dimethylsulfoxide and acetone, or equal amounts of dimethylformamide and acetone, generally for a period of 2 hours to 72 hours, where the preferred conditions are dimethylsulfoxide at ambient temperature for 18 hours.

Compounds of the formula V may also be prepared from compounds of the formula I by reaction of an appropriately substituted epoxide of the formula VIII either neat or in the presence of a polar solvent including but not limited to dimethylformamide, dimethylsulfoxide, and tetrahydrofuran. The aforesaid reaction can be performed at temperatures ranging from 0° C. to 100° C. for a period of 2 to 72 hours, where the preferred conditions are dimethylforamide at 60° C. for 24 hours.

Scheme 2 refers to the preparation of compounds of the formula V. Compounds of the formula V can be prepared from compounds of formula IX by reacting with a compound of formula XIV, H₂N—R¹, in the presence of a coupling reagent such as 1-[3-(dimethylamino)propyl]-3-ethylcarbodiimide (EDCI), dicyclohexylcarbodiimide (DCC), 1,1′-carbonyldiimidazole (CDI) and a base such as dimethylaminopyridine (DMAP) or triethylamine in an aprotic solvent, such as methylene chloride, dimethylformamide, or dimethylsulfoxide, preferably 1-[3-(dimethylamino)propyl]-3-ethylcarbodiimide and dimethylaminopyridine in dimethyl formamide. The aforesaid reaction may be run at a temperature from 22° C. to 60° C., for a period of 1 hour to 20 hours, preferably 22° C. for 18 hours.

Compounds of the formula V may also be prepared from compounds of the formula X by reaction by reacting with a compound of formula XIV in the presence of a base including but not limited to dimethylaminopyridine (DMAP), triethylamine, aqueous sodium hydroxide or aqueous potassium hydroxide in an aprotic solvent, such as methylene chloride, ethyl acetate, dichloroethane, dimethylformamide, or dimethylsulfoxide, preferably aqueous sodium hydroxide and dichloroethane. The aforesaid reaction may be run at a temperature from 22° C. to 60° C., for a period of 1 hour to 24 hours, preferably at ambient temperature for 3 hours. Compound X can be prepared from compound IX by reaction with a reagent capable of generating an acid chloride such as thionyl chloride or oxalyl chloride in the presence of a polar aprotic solvent such as ethyl acetate, methylene chloride, or dichloroethane at a temperature of 22° C. to 60° C., for a period of 1 hour to 24 hours, preferably oxalyl chloride in methylene chloride at ambient temperature for 16 hours.

Scheme 3 refers to the preparation of compounds of the formula IX, which can be converted into compounds of formula V by the methods described in Scheme 2. Compounds of formula IX can be prepared from compounds of formula XI using decarboxylation conditions, preferably mercaptoacetic acid in water containing a base such as sodium hydroxide at a temperature from 22° C. to 160° C. for a period of 1 hour to 24 hours, preferably 100° C. for 18 hours.

Scheme 4 refers to the preparation of compounds of the formula XIII and XI. Compounds of the formula XI can be converted into compounds of the formula IX by the methods described in Scheme 3.

A compound of formula XI can be prepared from a compound of formula XIII, wherein R⁵ is a suitable alkyl (C₁-C₂), by reaction with an acid such as 50% sulfuric acid at a temperature between 60° C. and 120° C., generally for a period between 30 minutes and 6 hours, preferably 2 hours at 120° C.

A compound of the formula XIII, wherein R⁸ is a suitable alkyl (C₁-C₂), can be prepared from the diazonium intermediate derived from a compound of formula XII. The diazonium intermediate is prepared by reaction of a compound of the formula XII with an acid such as hydrochloric acid and/or glacial acetic acid, followed by treatment with sodium nitrite in a solvent such as water at a temperature from 0° C. to 25° C., and the reaction is generally run from a period of 30 minutes to about 2 hours, preferably 10° C. for 30 minutes. A compound of the formula XII is prepared by the reaction of the above diazonium intermediate with a compound of the formula XVII: R⁸O(C═O)N(C═O)CH₂(C═O)N(C═O)OR⁸, under basic conditions. The reaction is typically carried out with sodium acetate as the base at a temperature from 0° C. to 120° C., preferably 10° C., then warmed to 120° C., and the reaction is generally run for a period of 1 hour to 24 hours, preferably 4 hours (Carrool, R. D.; et. al.; J. Med. Chem., 1983, 26, 96-100).

The activity of the P2X₇ receptor inhibiting compounds of the invention for the various disorders described above can be determined according to one or more of the following assays. All of the compounds of the invention that were tested had an IC₅₀ of less than 10 μM in the in vitro assay described below.

Preferably, the P2X₇ receptor inhibiting compounds of the invention have an IC₅₀ in the in vitro assays described below of less than 100 nM, more preferably less than 50 nM, and most preferably less than 10 nM. Still further, the compounds of the invention preferably have an IC₅₀ in the range of 0.01 nM-100 nM, more preferably between 0.05 nM-50 nM, and most preferably between 0.10 nM-10 nM.

Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3), p. 126). Consequently, when the receptor is activated using bbATP in the presence of ethidium bromide (a fluorescent DNA probe), an increase in the fluorescence of intracellular DNA-bound ethidium bromide is observed. Alternatively, the propidium dye YOPRO-1 can be substituted for ethidium bromide so as to detect uptake of the dye. The increase in fluorescence can be used as a measure of P2X₇ receptor activation and therefore to quantify the effect of a compound on the P2X₇ receptor.

In this manner, the compounds of the invention can be tested for antagonist activity at the P2X₇ receptor. 96-Well flat bottomed microtitre plates are filled with 250 μl of test solution comprising 200 μl of a suspension of THP-1 cells (2.5×10⁶ cells/ml, more preferably prestimulated as described in the literature with a combination of LPS and TNF to promote receptor expression) containing 10⁻⁴M ethidium bromide, 25 μl of a high potassium, low sodium buffer solution (10 mM Hepes, 150 mM KCl, 5 mM D-glucose and 1.0% FBS at pH 7.5) containing 10⁻⁵M bbATP, and 25 μl of the high potassium buffer solution containing 3×10⁻⁵M test compound (more preferably 5×10⁻⁴M, more preferably 1×10⁴M. more preferably 1×10⁻³M). The plate is covered with a plastic sheet and incubated at 37° C. for one hour. The plate is then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) can be used separately in the test as controls. From the readings obtained, a pIC₅₀ figure can be calculated for each test compound, this figure being the negative logarithm of the concentration of test compound necessary to reduce the bbATP agonist activity by 50%.

In like manner, the P2X₇ receptor inhibiting compounds of the invention can be tested for antagonist activity at the P2X₇ receptor using the cytokine IL-1β as the readout. Blood collected from normal volunteers in the presence of heparin is fractionated using lymphocyte separation medium obtained from Organon Technica (Westchester, Pa.). The region of the resulting gradient containing banded mononuclear cells is harvested, diluted with 10 ml of Maintenance Medium (RPMI 1640, 5% FBS, 25 mM Hepes, pH 7.2, 1% penicillin/streptomycin), and cells are collected by centrifugation. The resulting cell pellet was suspended in 10 ml of Maintenance Medium and a cell count was performed. In an average experiment, 2×10⁵ mononuclear cells are seeded into each well of 96-well plates in a total volume of 0.1 ml. Monocytes are allowed to adhere for 2 hours, after which the supernatants are discarded and the attached cells are rinsed twice and then incubated in Maintenance Medium overnight at 37° C. in a 5% CO₂ environment.

The cultured monocytes can be activated with 10 ng/ml LPS (E. coli serotype 055:B5; Sigma Chemicals, St. Louis, Mo.). Following a 2-hour incubation, the activation medium is removed, the cells are rinsed twice with 0.1 ml of Chase Medium (RPMI 1640, 1% FBS, 20 mM Hepes, 5 mM NaHCO₃, pH 6.9), and then 0.1 ml of Chase Medium containing a test agent is added and the plate is incubated for 30 minutes; each test agent concentration can be evaluated in triplicate wells. ATP then is introduced (from a 100 mM stock solution, pH 7) to achieve a final concentration of 2 mM and the plate is incubated at 37° C. for an additional 3 hours. Media were harvested and clarified by centrifugation, and their IL-1β content was determined by ELISA (R&D Systems; Minneapolis, Minn.).

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous), topical or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The compounds of formula I can also be formulated for sustained delivery according to methods well known to those of ordinary skill in the art. Examples of such formulations can be found in U.S. Pat. Nos. 3,538,214, 4,060,598, 4,173,626, 3,119,742, and 3,492,397, which are herein incorporated by reference in their entirety.

The active P2X₇ receptor inhibiting compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution, dry powder formulation or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, heptafluoroalkanes, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (inflammation) is 0.1 to 200 mg of the active ingredient per unit dose which could be administered, for example, 1 to 4 times per day.

The compound of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.10 to 70% w, of active ingredient, and, from 1 to 99.95% w, more preferably from 30 to 99.90% w, of a pharmaceutically acceptable adjuvant, diluent or carrier, all percentages by weight being based on total composition.

Aerosol formulations of the P2X₇ inhibiting compounds described herein for treatment of the conditions referred to above in the average adult human are preferably arranged so that each metered dose or “puff”of aerosol contains 20 μg to 1000 μg of the compound of the invention. The overall daily dose with an aerosol will be within the range 100 μg to 10 mg. Administration may be several times daily, for example 2, 3, 4 or 8 times, giving for example, 1, 2 or 3 doses each time.

Aerosol combination formulations for treatment of the conditions referred to above (e.g., adult respiratory distress syndrome) in the average adult human are preferably arranged so that each metered dose or “puff” of aerosol contains from about 1 μg to 1000 μg of the compound of the invention. The overall daily dose with an aerosol will be within the range 100 μg to 10 mg. Administration may be several times daily, for example 2, 3, 4 or 8 times, giving for example, 1, 2 or 3 doses each time.

Aerosol formulations for treatment of the conditions referred to above (e.g., adult respiratory distress syndrome) in the average adult human are preferably arranged so that each metered dose or “puff” of aerosol contains from about 20 μg to 1000 μg of the compound of the invention. The overall daily dose with an aerosol will be within the range 100 μg to 10 mg of the P2X₇ receptor inhibitor. Administration may be several times daily, for example 2, 3, 4 or 8 times, giving for example; 1, 2 or 3 doses each time.

This invention also encompasses the use in the methods and pharmaceutical combinations herein of pharmaceutical compositions containing prodrugs of compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues which are covalently joined through peptide bonds to free amino, hydroxy or carboxylic acid groups of compounds of formula I. The amino acid residues include the 20 naturally occurring amino acids commonly designated by three letter symbols and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters which are covalently bonded to the above substituents of formula I through the carbonyl carbon prodrug sidechain.

The following Examples illustrate the preparation of the P2X₇ receptor inhibiting compounds useful in the methods and combinations of the present invention. Melting points are uncorrected. NMR data are reported in parts per million (d) and are referenced to the deuterium lock signal from the sample solvent (deuteriochloroform unless otherwise specified). Mass Spectral data were obtained using a Micromass ZMD APCI Mass Spectrometer equipped with a Gilson gradient high performance liquid chromatograph. The following solvents and gradients were used for the analysis. Solvent A; 98% water/2% acetonitrile/0.01% formic acid and solvent B; acetonitrile containing 0.005% formic acid. Typically, a gradient was run over a period of about 4 minutes starting at 95% solvent A and ending with 100% solvent B. The mass spectrum of the major eluting component was then obtained in positive or negative ion mode scanning a molecular weight range from 165 AMU to 1100 AMU. Specific rotations were measured at room temperature using the sodium D line (589 nm). Commercial reagents were utilized without further purification. THF refers to tetrahydrofuran. DMF refers to N,N-dimethylformamide. Chromatography refers to column chromatography performed using 32-63 mm silica gel and executed under nitrogen pressure (flash chromatography) conditions. Room or ambient temperature refers to 20-25° C. All non-aqueous reactions were run under a nitrogen atmosphere for convenience and to maximize yields. Concentration at reduced pressure means that a rotary evaporator was used.

One of ordinary skill in the art will appreciate that in some cases protecting groups may be required during preparation. After the target molecule is made, the protecting group can be removed by methods well known to those of ordinary skill in the art, such as described in Greene and Wuts, “Protective Groups in Organic Synthesis” (3rd Ed, John Wiley & Sons 1999).

### EXAMPLE 1

### 5-(4-Carbamoylmethyl-3,5-dioxo-4,5-dihydro-3H-[1,2,4]triazin-2-yl)-N-(1-hydroxy-cycloheptylmethyl)-2-methyl-benzamide

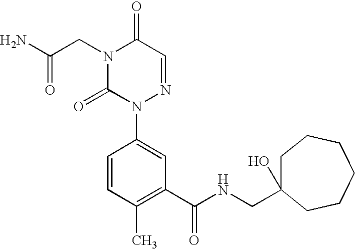

### (A) 5-Amino-2-methyl-benzoic acid hydrochloride salt

A slurry of 5-nitro-2-methyl-benzoic acid (17.1 g, 94.4 mmol) and 10% Pd/C (500 mg) in EtOH (500 mL) was shaken under 40 psi H₂ at ambient temperature for 4 hours. HCl was added and the solution filtered through a pad of celite. The filtrate was concentrated in vacuo to give the title compound (17.2 g).

### (B) 2-(3-Carboxy-4-methyl-phenyl)-3,5-dioxo-2,3,4,5-tetrahydro-[1,2,4]triazine-6-carboxylic acid

To a solution of 5-Amino-2-methyl-benzoic acid hydrochloride salt (15.2 g, 81.2 mmol) in acetic acid (300 mL) was added concentrated HCl (21.0 mL). The resulting slurry was stirred at ambient temperature for 30 minutes. The reaction was then cooled to 10° C., and a solution of sodium nitrite (6.17 g, 89.4 mmol) in water (15 mL) was added dropwise. The reaction was stirred at 10° C. for 30 minutes, when sodium acetate (14.7 g, 179.0 mmol) and (3-ethoxycarbonylamino-3-oxo-propionyl)-carbamic acid ethyl ester (J. Chem. Soc. Perkins Trans. 1, 1991, 2317) (22.0 g, 89.4 mmol) were added. The reaction was let stir at 10° C. for 20 minutes, then warmed to room temperature and stirred for 1 hour. Sodium acetate (6.7 g, 81.2 mmol) was then added and the reaction refluxed for 14 hours. A 50% aqueous solution of H₂SO₄ (88.0 mL) was added and the reaction refluxed for 2 hours. The reaction was cooled, then water (50 mL) added. The resulting tan precipitate was filtered, washed with water, and dried to give the title compound (17.8 g).

### (C) 5-(3,5-Dioxo-4,5-dihydro-3H-[1,2,4]triazin-2-yl)-2-methyl-benzoic acid

2-(3-Carboxy-4-methyl-phenyl)-3,5-dioxo-2,3,4,5-tetrahydro-[1,2,4]triazine-6-carboxylic acid (110 gm) was added to 8 volumes of water with 2.4 equivalents of sodium hydroxide and 1.1 equivalents of mercaptoacetic acid. The reaction mixture was heated to reflux (100-105° C.) for approximately 18 hours at which point the reaction was complete by HPLC. 30% Sodium hydroxide and toluene were added and the resulting mixture was stirred. Upon settling a large interface was noted. More water, toluene and some ethyl acetate were added. The interface was minimized. The water layer was separated and treated with 2N HCl. At pH 2 solids precipitated out and the slurry was cooled to <10° C. The solids were filtered off in a slow filtration and dried in a vacuum oven to give 69 gm of the title compound.

### (D) 5-(3,5-Dioxo-4,5-dihydro-3H-[1,2,4]triazin-2-yl)-N-(1-hydroxy-cycloheptylmethyl)-2-methyl-benzamide

A slurry of 5-(3,5-Dioxo-4,5-dihydro-3H-[1,2,4]triazin-2-yl)-2-methyl-benzoic acid (5.0 g, 20.2 mmol), 1-aminomethyl-cycloheptanol HCl (5.4 g, 30.3 mmol), EDCI (5.8 g), and DMAP (7.4 g, 60.6 mmol) in DMF (67.3 mL) was stirred at ambient temperature for 14 hours. The reaction was then poured into 1N HCl (50 mL) and diluted with water (15 fold). The aqueous was extracted with CH₂Cl₂ (3×). The organics were combined, washed with brine, dried over sodium sulfate, and concentrated in vacuo to give a tan solid. The crude was recrystallized from CH₂Cl₂ to give the title compound as an off-white solid (3.1 g).

### (E) 5-(4-Carbamoylmethyl-3,5-dioxo-4,5-dihydro-3H-[1,2,4]triazin-2-yl)-N-(1-hydroxy-cycloheptylmethyl)-2-methyl-benzamide

A slurry of 5-(3,5-Dioxo-4,5-dihydro-3H-[1,2,4]triazin-2-yl)-N-(1-hydroxy-cycloheptylmethyl)-2-methyl-benzamide (200.0 mg, 0.537 mmol) and Cs₂CO₃ (290.3 mg, 0.891 mmol) were stirred in DMSO (1.79 mL, 0.3 M) at ambient temperature for 15 minutes. 2-Bromoacetamide (74.1 mg, 0.537 mmol) was added and the reaction stirred at ambient temperature for 14 hours. The reaction was diluted with water (15-fold) and the aqueous extracted with CH₂Cl₂ (3×). The organics were dried over sodium sulfate, and concentrated in vacuo to a tan oil. The crude was triturated from IPE/Et₂O/CH₂Cl₂ to give the title compound as a tan solid (105 mg). LCMS (m/z) 430.5 M+1.

The compounds of Examples 2-44, identified in Table 1 below, can be prepared according to the method of Example 1.

TABLE 1

| EXAMPLE | STRUCTURE | NAME | DATA LCMS M/Z |
|---|---|---|---|
| 2 | 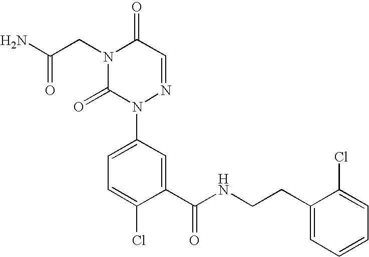 | 5-(4-Carbamoylmethyl- 3,5-dioxo-4,5-dihydro-3H- [1,2,4]triazin-2-yl)-2- chloro-N-[2-(2-chloro- phenyl)-ethyl]-benzamide | 462.1 |
| 3 | 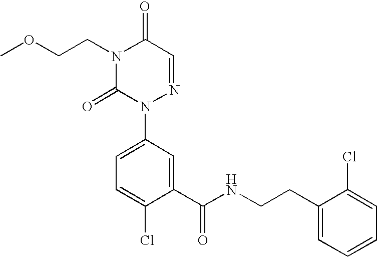 | 2-Chloro-N-[2-(2-chloro- phenyl)-ethyl]-5-[4-(2- methoxy-ethyl)-3,5-dioxo- 4,5-dihydro-3H- [1,2,4]triazin-2-yl]- benzamide | 463.2 |
| 4 | 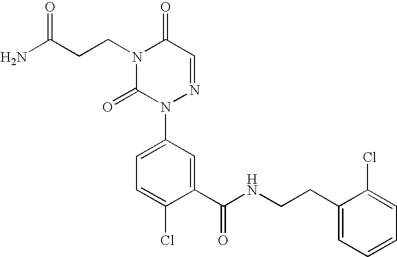 | 5-[4-(2-Carbamoyl-ethyl)- 3,5-dioxo-4,5-dihydro-3H- [1,2,4]triazin-2-yl]-2- chloro-N-[2-(2-chloro- phenyl)-ethyl]-benzamide | 476.3 |
| 5 | 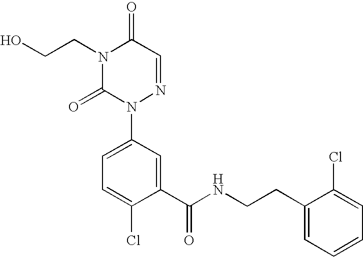 | 2-Chloro-N-[2-(2-chloro- phenyl)-ethyl]-5-[4-(2- hydroxy-ethyl)-3,5-dioxo- 4,5-dihydro-3H- [1,2,4]triazin-2-yl]- benzamide | 449.4 |
| 6 | 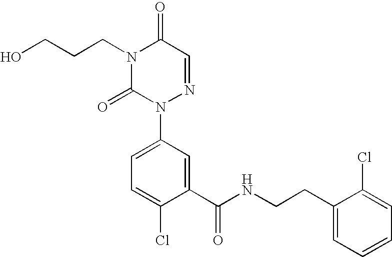 | 2-Chloro-N-[2-(2-chloro- phenyl)-ethyl]-5-[4-(3- hydroxy-propyl)-3,5- dioxo-4,5-dihydro-3H- [1,2,4]triazin-2-yl]- benzamide | 463.6 |
| 7 | 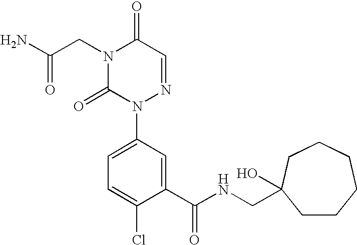 | 5-(4-Carbamoylmethyl- 3,5-dioxo-4,5-dihydro-3H- chloro-N-(1-hydroxy- cycloheptylmethyl)- benzamide | 450.9 |
| 8 | 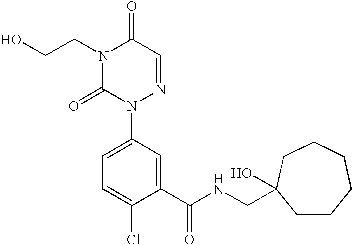 | 2-Chloro-N-(1-hydroxy- cycloheptylmethyl)-5-[4- (2-hydroxy-ethyl)-3,5- dioxo-4,5-dihydro-3H- [1,2,4]triazin-2-yl]- benzamide | 437.9 |
| 9 | 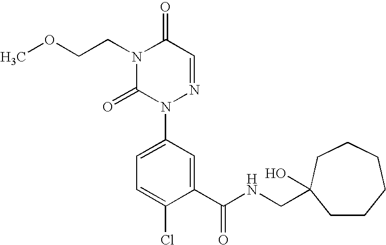 | 2-Chloro-N-(1-hydroxy- cycloheptylmethyl)-5-[4- (2-methoxy-ethyl)-3,5- dioxo-4,5-dihydro-3H- [1,2,4]triazin-2-yl]- benzamide | 451.7 |
| 10 | 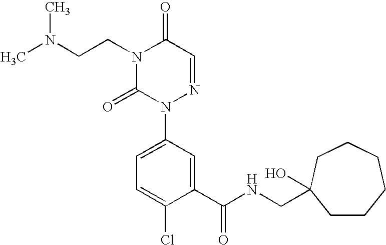 | 2-Chloro-5-[4-(2- dimethylamino-ethyl)-3,5- dioxo-4,5-dihydro-3H- [1,2,4]triazin-2-yl]-N-(1- hydroxy- cycloheptylmethyl)- benzamide | 464.5 |
| 11 | 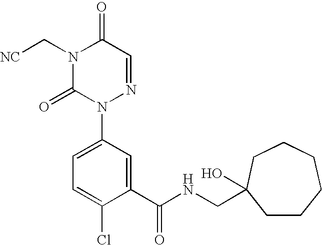 | 2-Chloro-5-(4- cyanomethyl-3,5-dioxo- 4,5-dihydro-3H- [1,2,4]triazin-2-yl)-N-(1- hydroxy- cycloheptylmethyl)- benzamide | 432.3 |
| 12 | 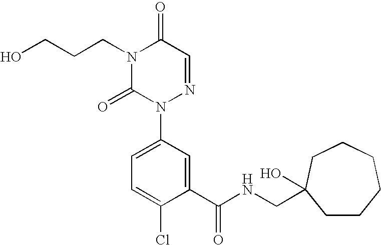 | 2-Chloro-N-(1-hydroxy- cycloheptylmethyl)-5-[4- (3-hydroxy-propyl)-3,5- dioxo-4,5-dihydro-3H- [1,2,4]triazin-2-yl]- benzamide | 451.4 |
| 13 | 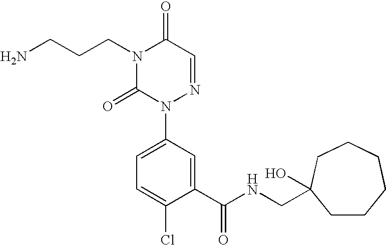 | 5-[4-(2-Amino-ethyl)-3,5- dioxo-4,5-dihydro-3H- [1,2,4]triazin-2-yl]-2- chloro-N-(1-hydroxy- cycloheptylmethyl)- benzamide | 436.5 |
| 14 | 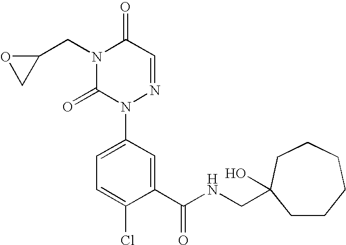 | 2-Chloro-N-(1-hydroxy- cycloheptylmethyl)-5-(4- oxiranylmethyl-3,5-dioxo- 4,5-dihydro-3H- [1,2,4]triazin-2-yl)- benzamide | 449.5 |
| 15 | 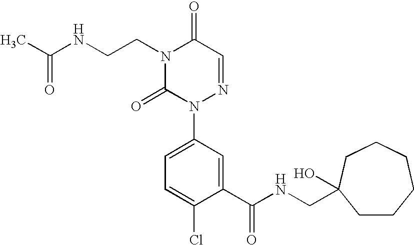 | 5-[4-(2-Acetylamino- ethyl)-3,5-dioxo-4,5- dihydro-3H-[1,2,4]triazin- 2-yl]-2-chloro-N-(1- hydroxy- cycloheptylmethyl)- benzamide | 478.4 |
| 16 | 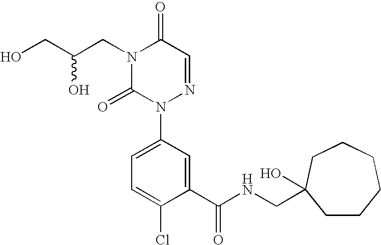 | 2-Chloro-5-[4-(2,3- dihydroxy-propyl)-3,5- dioxo-4,5-dihydro-3H- [1,2,4]triazin-2-yl]-N-(1- hydroxy- cycloheptylmethyl)- benzamide | 467.4 |
| 17 | 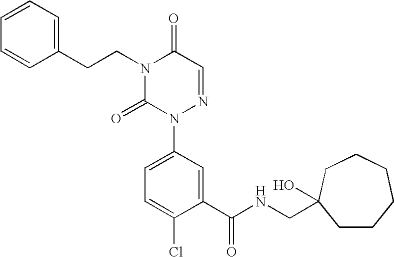 | 2-Chloro-5-(3,5-dioxo-4- phenethyl-4,5-dihydro-3H- [1,2,4]triazin-2-yl)-N-(1- hydroxy- cycloheptylmethyl)- benzamide | 497.5 |
| 18 | 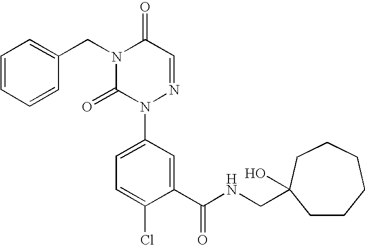 | 5-(4-Benzyl-3,5-dioxo-4,5- dihydro-3H-[1,2,4]triazin- 2-yl)-2-chloro-N-(1- hydroxy- cycloheptylmethyl)- benzamide | 483.5 |
| 19 | 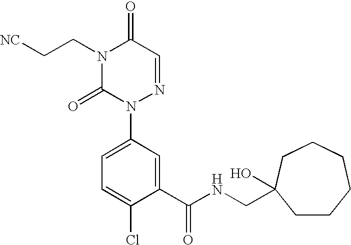 | 2-Chloro-5-[4-(2-cyano- ethyl)-3,5-dioxo-4,5- dihydro-3H-[1,2,4]triazin- 2-yl]-N-(1-hydroxy- cycloheptylmethyl)- benzamide | 446.5 |
| 20 | 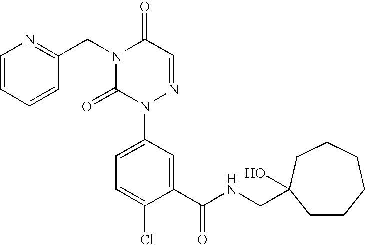 | 2-Chloro-5-(3,5-dioxo-4- pyridin-2-ylmethyl-4,5- dihydro-3H-[1,2,4]triazin- 2-yl)-N-(1-hydroxy- cycloheptylmethyl)- benzamide | 484.4 |
| 21 | 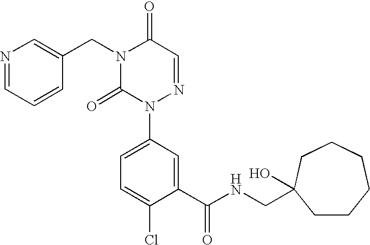 | 2-Chloro-5-(3,5-dioxo-4- pyridin-3-ylmethyl-4,5- dihydro-3H-[1,2,4]triazin- 2-yl)-N-(1-hydroxy- cycloheptylmethyl)- benzamide | 484.5 |
| 22 | 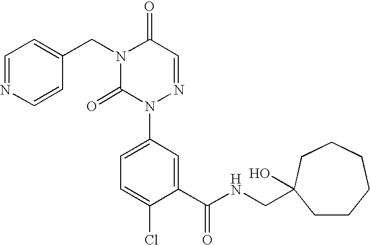 | 2-Chloro-5-(3,5-dioxo-4- pyridin-4-ylmethyl-4,5- dihydro-3H-[1,2,4]triazin- 2-yl)-N-(1-hydroxy- cycloheptylmethyl)- benzamide | 484.5 |
| 23 | 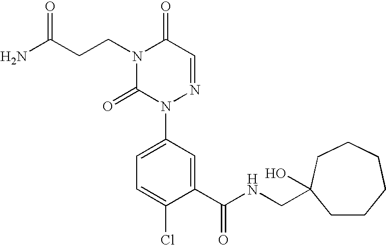 | 5-[4-(2-Carbamoyl-ethyl)- 3,5-dioxo-4,5-dihydro-3H- [1,2,4]triazin-2-yl]-2- chloro-N-(1-hydroxy- cycloheptylmethyl)- benzamide | 474.5 |
| 24 | 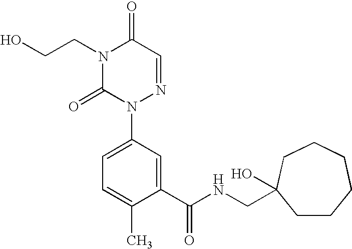 | N-(1-Hydroxy- cycloheptylmethyl)-5-[4- (2-hydroxy-ethyl)-3,5- dioxo-4,5-dihydro-3H- [1,2,4]triazin-2-yl]-2- methyl-benzamide | 417.5 |
| 25 | 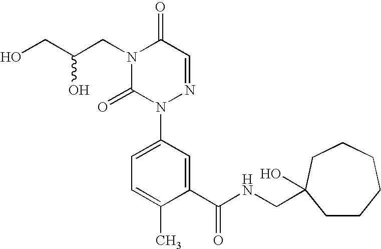 | 5-[4-(2,3-Dihydroxy- propyl)-3,5-dioxo-4,5- dihydro-3H-[1,2,4]triazin- 2-yl]-N-(1-hydroxy- cycloheptylmethyl)-2- methyl-benzamide | 447.3 |
| 26 | 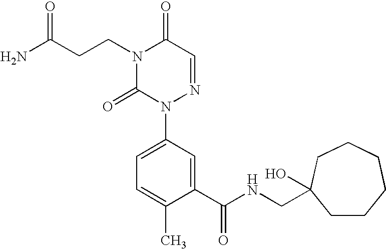 | 5-[4-(2-Carbamoyl-ethyl)- 3,5-dioxo-4,5-dibydro-3H- [1,2,4]triazin-2-yl]-N-(1- hydroxy- cycloheptylmethyl)-2- methyl-benzamide | 444.6 |
| 27 | 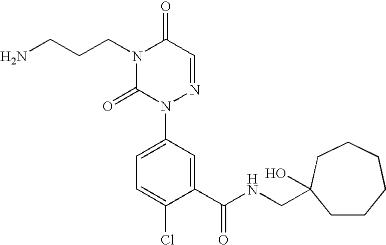 | 5-[4-(3-Amino-propyl)- 3,5-dioxo-4,5-dihydro-3H- [1,2,4]triazin-2-yl]-2- chloro-N-(1-hydroxy- cycloheptylmethyl)- benzamide | 450.4 |
| 28 | 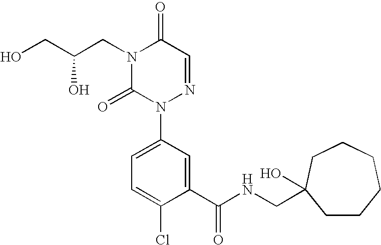 | 2-Chloro-5-[4-(2,3- dihydroxy-propyl)-3,5- dioxo-4,5-dihydro-3H- [1,2,4]triazin-2-yl]-N-(1- hydroxy- cycloheptylmethyl)- benzamide | 467.4 |
| 29 | 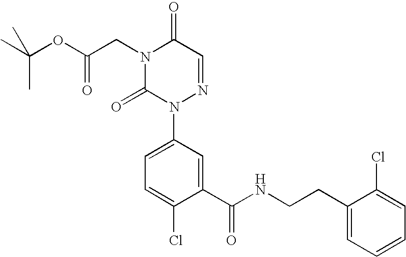 | (2-{4-Chloro-3-[2-(2- chloro-phenyl)- ethylcarbamoyl]-phenyl}- 3,5-dioxo-2,5-dihydro-3H- [1,2,4]triazin-4-yl)-acetic acid tert-butyl ester | 519.7 |
| 30 | 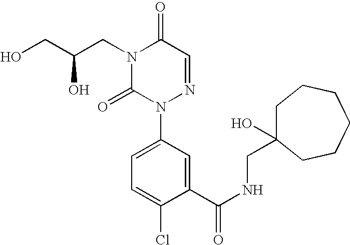 | 2-Chloro-5-[4-(2,3- dihydroxy-propyl)-3,5- dioxo-4,5-dihydro-3H- [1,2,4]triazin-2-yl]-N-(1- hydroxy- cycloheptylmethyl)- benzamide | 467.6 |
| 31 | 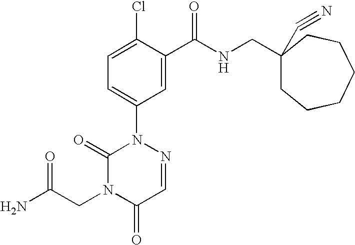 | 5-(4-Carbamoylmethyl- 3,5-dioxo-4,5-dihydro-3H- [1,2,4]triazin-2-yl)-2- chloro-N-(1-cyano- cycloheptylmethyl)- benzamide | 459.3 |
| 32 | 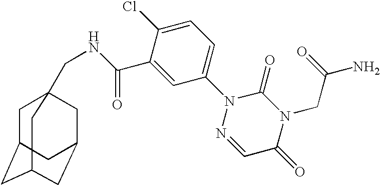 | N-Adamantan-1-ylmethyl- 5-(4-carbamoylmethyl-3,5- dioxo-4,5-dihydro-3H- [1,2,4]triazin-2-yl)-2- chloro-benzamide | 473.0 |
| 33 | 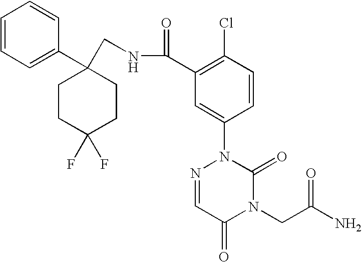 | 5-(4-Carbamoylmethyl- 3,5-dioxo-4,5-dihydro-3H- [1,2,4]triazin-2-yl)-2- chloro-N-(4,4-difluoro-1- benzamide | 532.3 |
| 34 | 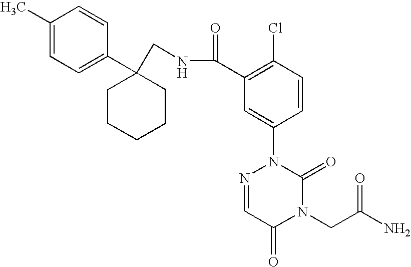 | 5-(4-Carbamoylmethyl- 3,5-dioxo-4,5-dihydro-3H- [1,2,4]triazin-2-yl)-2- chloro-N-(1-p-tolyl- cyclohexylmethyl)- benzamide | 510.4 |
| 35 | 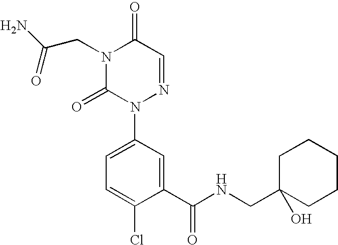 | 5-(4-Carbamoylmethyl- 3,5-dioxo-4,5-dihydro-3H- chloro-N-(1-hydroxy- cyclohexylmethyl)- benzamide | 436.5 |
| 36 | 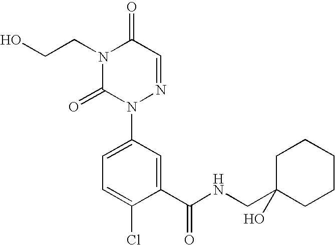 | 2-Chloro-N-(1-hydroxy- cyclohexylmethyl)-5-[4-(2- hydroxy-ethyl)-3,5- dioxo-4,5-dihydro-3H- [1,2,4]triazin-2-yl]- benzamide | 423.5 |
| 37 | 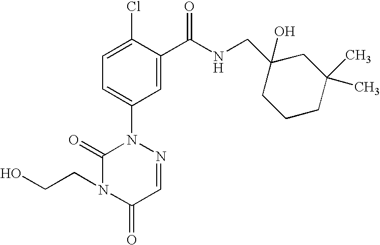 | 2-Chloro-N-(1-hydroxy- 3,3-dimethyl- cyclohexylmethyl)-5-[4-(2- hydroxy-ethyl)-3,5- dioxo-4,5-dihydro-3H- [1,2,4]triazin-2-yl]- benzamide | 451.5 |
| 38 | 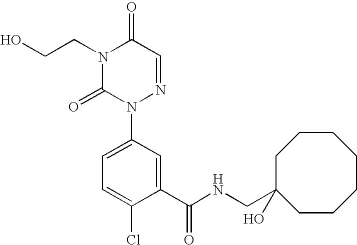 | 2-Chloro-N-(1-hydroxy- cyclooctylmethyl)-5-[4-(2- hydroxy-ethyl)-3,5-dioxo- 4,5-dihydro-3H- [1,2,4]triazin-2-yl]- benzamide | 451.5 |
| 39 | 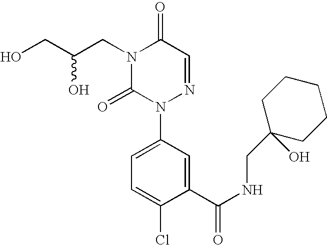 | 2-Chloro-5-[4-(2,3- dihydroxy-propyl)-3,5- dioxo-4,5-dihydro-3H- [1,2,4]triazin-2-yl]-N-(1- hydroxy- cyclohexylmethyl)- benzamide | 453.5 |
| 40 | 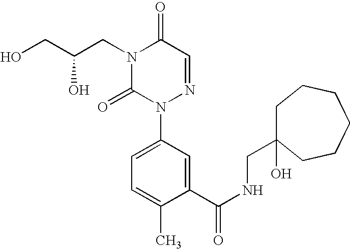 | 5-[4-(2,3-Dihydroxy- propyl)-3,5-dioxo-4,5- dihydro-3H-[1,2,4]triazin- 2-yl]-N-(1-hydroxy- cycloheptylmethyl)-2- methyl-benzamide | 447.6 |
| 41 | 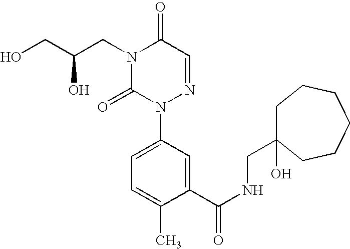 | 5-[4-(2,3-Dihydroxy- propyl)-3,5-dioxo-4,5- dihydro-3H-[1,2,4]triazin- 2-yl]-N-(1-hydroxy- cycloheptylmethyl)-2- methyl-benzamide | 447.6 |
| 42 | 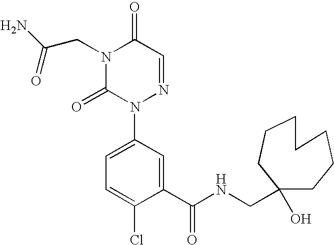 | 5-(4-Carbamoylmethyl- 3,5-dioxo-4,5-dihydro-3H- [1,2,4]triazin-2-yl)-2- chloro-N-(1-hydroxy- cyclooctylmethyl)- benzamide | 462.4 (M − 1) |
| 43 | 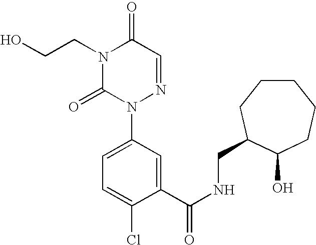 | 2-Chloro-N-(2-hydroxy- cycloheptylmethyl)-5-[4- (2-hydroxy-ethyl)-3,5- dioxo-4,5-dihydro-3H- [1,2,4]triazin-2-yl]- benzamide | 437.3 |
| 44 | 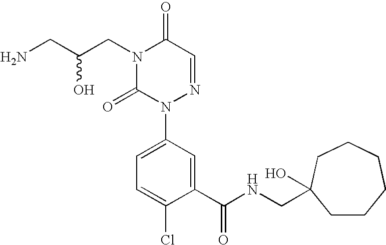 | 5-[4-(3-Amino-2-hydroxy- propyl)-3,5-dioxo-4,5- dihydro-3H-[1,2,4]triazin- 2-yl]-2-chloro-N-(1- hydroxy- cycloheptylmethyl)- benzamide | 448.5 |

### EXAMPLE 45

### 2-Chlore-N-[2-(2-chloro-phenyl)-ethyl]-5-(4-methylcarbamoylmethyl-3,5-dioxo-4,5-dihydro-3H-[1,2,4]triazin-2-yl)-benzamide

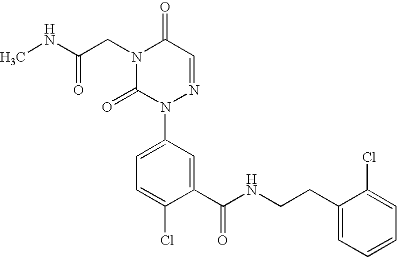

### (A) (2-{4-Chloro-3-[2-(2-chloro-phenyl)-ethylcarbamoyl]-phenyl}-3,5-dioxo-2,5-dihydro-3H-[1,2,4]triazin-4-yl)-acetic acid

A solution of Example 34 (358 mg, 0.69 mmol) and TFA (1 mL) was stirred at ambient temperature for 18 hours. The solvent was removed in vacuo, and excess TFA azeotroped using CH₂Cl₂ (3×). The crude pale brown solid was triterated in hexane to give the title compound (295 mg).

### (B) 2-Chloro-N-[2-(2-chloro-phenyl)-ethyl]-5-(4-methylcarbamoylmethyl-3,5-dioxo-4,5-dihydro-3H-[1,2,4]triazin-2-yl)-benzamide

A slurry of (2-{4-Chloro-3-[2-(2-chloro-phenyl)-ethylcarbamoyl]-phenyl}-3,5-dioxo-2,5-dihydro-3H-[1,2,4]triazin-4-yl)-acetic acid (71.4 mg, 0.154 mmol), methylamine HCl (15.6 mg, 0.231 mmol), EDCI (44.4 mg, 0.231 mmol), and DMAP (75.5 mg, 0.616 mmol) in DMF (1.0 mL) were stirred at ambient temperature for 20 hours. The reaction was diluted with 1N HCl, and let stir for 5 hours. The crude was filtered and triturated from hexane to give the title compound (20 mg). LCMS (m/z) 476.1 M+1.

The compounds of Examples 46-60, identified in Table 2 below, can be prepared according to the method of Example 45.

TABLE 2

| EXAMPLE | STRUCTURE | NAME | DATA LCMS M/Z |
|---|---|---|---|
| 46 | 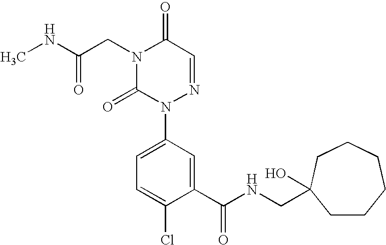 | 2-Chloro-N-(1-hydroxy- cycloheptylmethyl)-5-(4- methylcarbamoylmethyl- 3,5-dioxo-4,5-dihydro-3H- [1,2,4]triazin-2-yl)- benzamide | 464.8 |
| 47 | 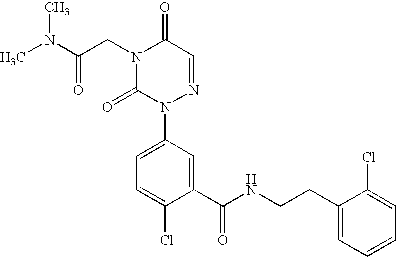 | 2-Chloro-N-[2-(2-chloro- phenyl)-ethyl]-5-(4- dimethylcarbamoylmethyl- 3,5-dioxo-4,5-dihydro-3H- [1,2,4]triazin-2-yl)- benzamide | 490.1 |
| 48 | 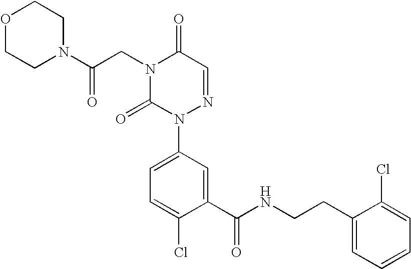 | 2-Chloro-N-[2-(2-chloro- phenyl)-ethyl]-5-[4-(2- morpholin-4-yl-2-oxo- ethyl)-3,5-dioxo-4,5- dihydro-3H-[1,2,4]triazin- 2-yl]-benzamide | 523.3 |
| 49 | 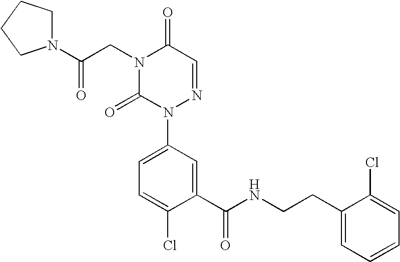 | 2-Chloro-N-[2-(2-chloro- phenyl)-ethyl]-5-[3,5- dioxo-4-(2-oxo-2- pyrrolidin-1-yl-ethyl)-4,5- dihydro-3H-[1,2,4]triazin- 2-yl]-benzamide | 516.3 |
| 50 | 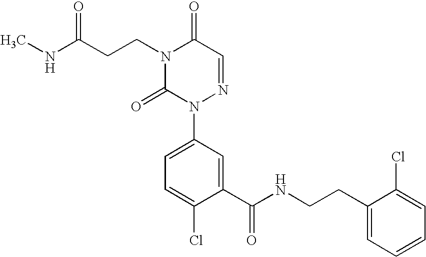 | 2-Chloro-N-[2-(2-chloro- phenyl)-ethyl]-5-[4-(2- methylcarbamoyl-ethyl)- 3,5-dioxo-4,5-dihydro-3H- [1,2,4]triazin-2-yl]- benzamide | 490.9 |
| 51 | 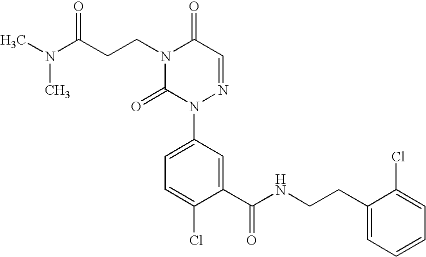 | 2-Chloro-N-[2-(2-chloro- phenyl)-ethyl]-5-[4-(2- dimethylcarbamoyl-ethyl)- 3,5-dioxo-4,5-dihydro-3H- [1,2,4]triazin-2-yl]- benzamide | 504.5 |
| 52 | 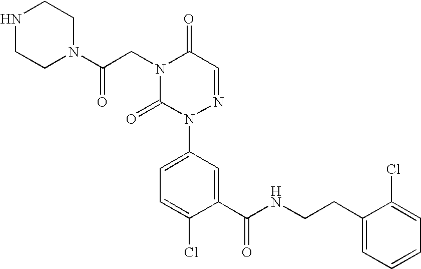 | 2-Chloro-N-[2-(2-chloro- phenyl)-ethyl]-5-[3,5- dioxo-4-(2-oxo-2-piperazin- 1-yl-ethyl)-4,5-dihydro-3H- [1,2,4]triazin-2-yl]- benzamide | 531.5 |
| 53 | 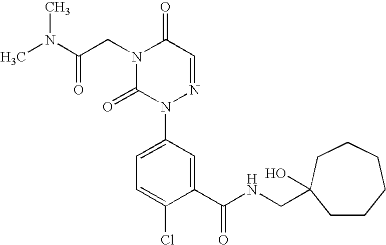 | 2-Chloro-5-(4- dimethylcarbamoylmethyl- 3,5-dioxo-4,5-dihydro-3H- [1,2,4]triazin-2-yl)-N-(1- hydroxy- cycloheptylmethyl)- benzamide | 478.8 |
| 54 | 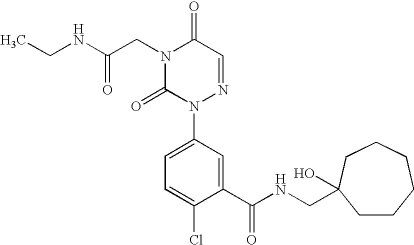 | 2-Chloro-5-(4- ethylcarbamoylmethyl-3,5- dioxo-4,5-dihydro-3H- [1,2,4]triazin-2-yl)-N-(1- hydroxy- cycloheptylmethyl)- benzamide | 478.8 |
| 55 | 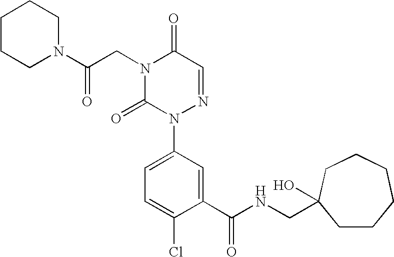 | 2-Chloro-5-[3,5-dioxo-4-(2- oxo-2-piperidin-1-yl-ethyl)- 4,5-dihydro-3H- [1,2,4]triazin-2-yl]-N-(1- hydroxy- cycloheptylmethyl)- benzamide | 518.8 |
| 56 | 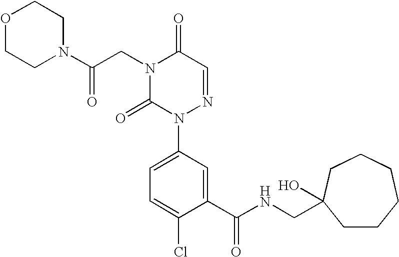 | 2-Chloro-N-(1-hydroxy- cycloheptylmethyl)-5-[4-(2- morpholin-4-yl-2-oxo- ethyl)-3,5-dioxo-4,5- dihydro-3H-[1,2,4]triazin- 2-yl]-benzamide | 520.8 |
| 57 | 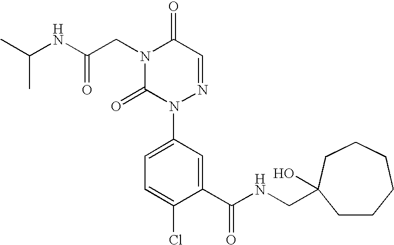 | 2-Chloro-N-(1-hydroxy- cycloheptylmethyl)-5-[4- (isopropylcarbamoyl- methyl)-3,5-dioxo-4,5- dihydro-3H-[1,2,4]triazin- 2-yl]-benzamide | 492.8 |
| 58 | 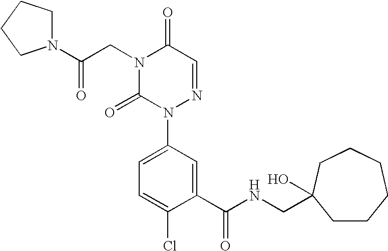 | 2-Chloro-5-[3,5-dioxo-4-(2- oxo-2-pyrrolidin-1-yl- ethyl)-4,5-dihydro-3H- [1,2,4]triazin-2-yl]-N-(1- hydroxy- cycloheptylmethyl)- benzamide | 504.4 |
| 59 | 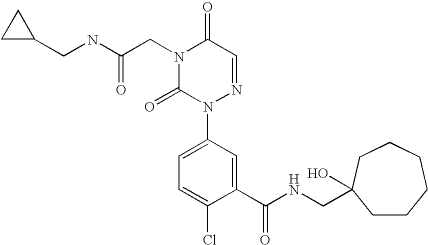 | 2-Chloro-5-{4- [(cyclopropylmethyl- carbamoyl)-methyl]-3,5- dioxo-4,5-dihydro-3H- [1,2,4]triazin-2-yl}-N-(1- hydroxy- cycloheptylmethyl)- benzamide | 504.4 |
| 60 | 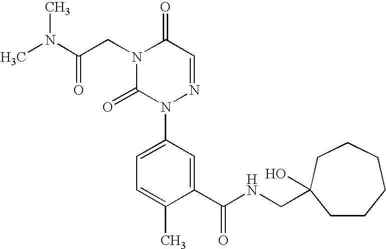 | 5-(4- Dimethylcarbamoylmethyl- 3,5-dioxo-4,5-dihydro-3H- [1,2,4]triazin-2-yl)-N-(1- hydroxy- cycloheptylmethyl)-2- methyl-benzamide | 458.5 |

### EXAMPLE 61

### 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide

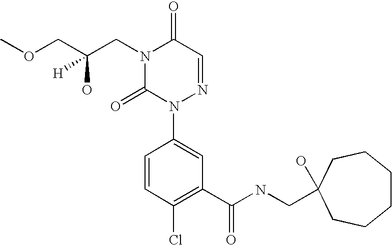

### (A) 2-Chloro-N-(1-hydroxy-cycloheptylmethyl)-5-[4-(2R-hydroxy-3-methoxy-propyl)-3,5-dioxo-4,5-dihydro-3H-[1,2,4]triazin-2-yl]-benzamide

5-(3,5-Dioxo-4,5-dihydro-3H-[1,2,4]triazin-2-yl)-N-(1-hydroxy-cycloheptylmethyl)-2-chloro-benzamide (1.77 g, 4.5 mmol) and R-(−)-glycidyl methyl ether (2.5 mL, 27.8 mmol) in DMF (4.5 mL) were heated at 60° C. for 18 hours. The reaction was cooled, diluted with 1N HCl, and extracted with CH₂Cl₂. The organics were combined, washed with sat'd sodium bicarbonate, dried over sodium sulfate and charcoal, filtered, and concentrated in vacuo. The crude was purified by silica gel flash chromatography (elution with EtOAc), then recrystallized from ethyl acetate/hexane to give the title compound (1.62 g). LCMS (m/z) 479.5 M−1.

The compounds of Examples 62-99, identified in Table 3 below, can be prepared according to the method of Example 61.

TABLE 3

| | | | DATA LCMS |
|---|---|---|---|
| EXAMPLE | STRUCTURE | NAME | M/Z |
| 62 | 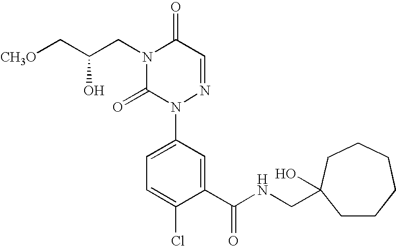 | 2-Chloro-N-(1-hydroxy- cycloheptylmethyl)-5-[4- (2-hydroxy-3-methoxy- propyl)-3,5-dioxo-4,5- dihydro-3H-[1,2,4]triazin- 2-yl]-benzamide | 481.5 |
| 63 | 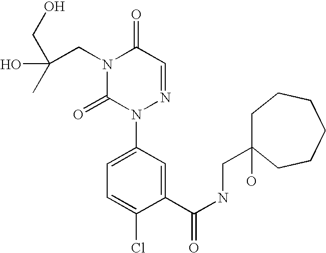 | 2-Chloro-5-[4-(2,3- dihydroxy-2-methyl- propyl)-3,5-dioxo-4,5- dihydro-3H-[1,2,4]triazin- 2-yl]-N-(1-hydroxy- cycloheptylmethyl)- benzamide | 481.6 |
| 64 | 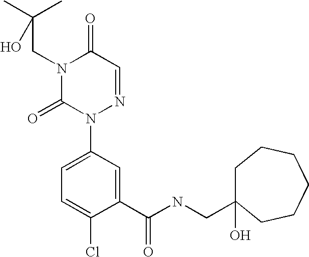 | 2-Chloro-N-(1-hydroxy- cycloheptylmethyl)-5-[4- (2-hydroxy-2-methyl- propyl)-3,5-dioxo-4,5- dihydro-3H-[1,2,4]triazin- 2-yl]-benzamide | 465.5 |
| 65 | 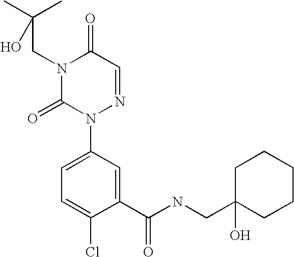 | 2-Chloro-N-(1-hydroxy- cyclohexylmethyl)-5-[4- (2-hydroxy-2-methyl- propyl)-3,5-dioxo-4,5- dihydro-3H-[1,2,4]triazin- 2-yl]-benzamide | 451.5 |
| 66 | 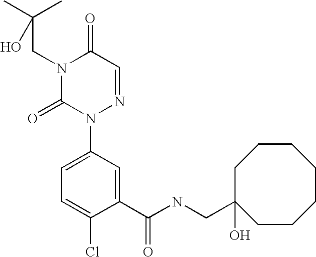 | 2-Chloro-N-(1-hydroxy- cyclooctylmethyl)-5-[4- (2-hydroxy-2-methyl- propyl)-3,5-dioxo-4,5- dihydro-3H-[1,2,4]triazin- 2-yl]-benzamide | 479.4 |
| 67 | 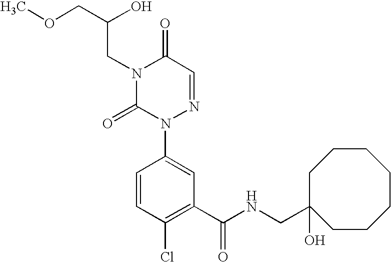 | 2-Chloro-N-(1-hydroxy- cyclooctylmethyl)-5-[4- (2-hydroxy-3-methoxy- propyl)-3,5-dioxo-4,5- dihydro-3H-[1,2,4]triazin- 2-yl]-benzamide | 495.6 |
| 68 | 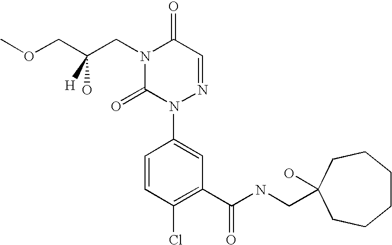 | 2-Chloro-N-(1-hydroxy- cycloheptylmethyl)-5-[4- (2-bydroxy-3-methoxy- propyl)-3,5-dioxo-4,5- dihydro-3H-[1,2,4]triazin- 2-yl]-benzamide | 479.4 (M − 1) |
| 69 | 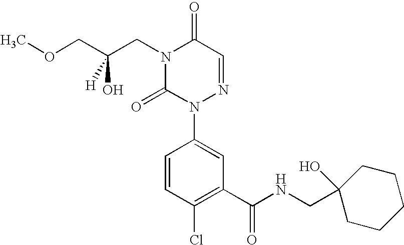 | 2-Chloro-N-(1-hydroxy- cyclohexylmethyl)-5-[4- (2-hydroxy-3-methoxy- propyl)-3,5-dioxo-4,5- dihydro-3H-[1,2,4]triazin- 2-yl]-benzamide | 467.5 |
| 70 | 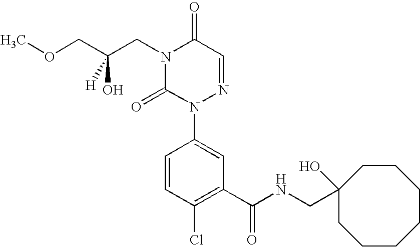 | 2-Chloro-N-(1-hydroxy- cyclooctylmethyl)-5-[4- (2-hydroxy-3-methoxy- propyl)-3,5-dioxo-4,5- dihydro-3H-[1,2,4]triazin- 2-yl]-benzamide | 495.6 |
| 71 | 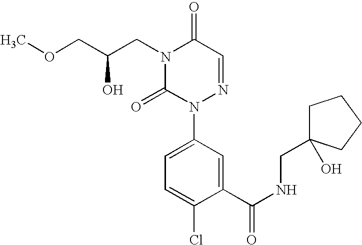 | 2-Chloro-N-(1-hydroxy- cyclopentylmethyl)-5-[4- (2-hydroxy-3-methoxy- propyl)-3,5-dioxo-4,5- dihydro-3H-[1,2,4]triazin- 2-yl]-benzamide | 453.5 |
| 72 | 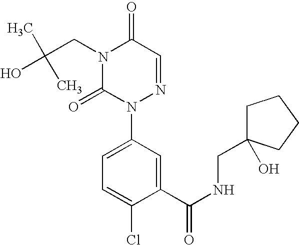 | 2-Chloro-N-(1-hydroxy- cyclopentylmethyl)-5-[4- (2-hydroxy-2-methyl- propyl)-3,5-dioxo-4,5- dihydro-3H-[1,2,4]triazin- 2-yl]-benzamide | 437.4 |
| 73 | 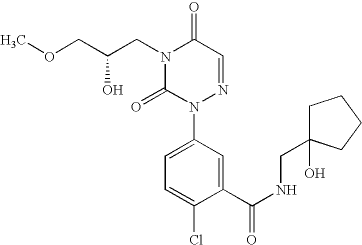 | 2-Chloro-N-(1-hydroxy- cyclopentylmethyl)-5-[4- (2-hydroxy-3-methoxy- propyl)-3,5-dioxo-4,5- dihydro-3H-[1,2,4]triazin- 2-yl]-benzamide | 453.5 |
| 74 | 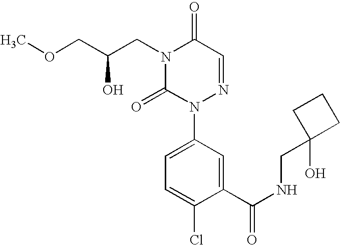 | 2-Chloro-N-(1-hydroxy- cyclobutylmethyl)-5-[4- (2-hydroxy-3-methoxy- propyl)-3,5-dioxo-4,5- dihydro-3H-[1,2,4]triazin- 2-yl]-benzamide | 439.5 |
| 75 | 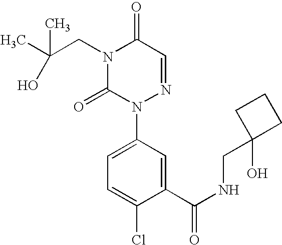 | 2-Chloro-N-(1-hydroxy- cyclobutylmethyl)-5-[4- (2-hydroxy-2-methyl- propyl)-3,5-dioxo-4,5- dihydro-3H-[1,2,4]triazin- 2-yl]-benzamide | 423.3 |
| 76 | 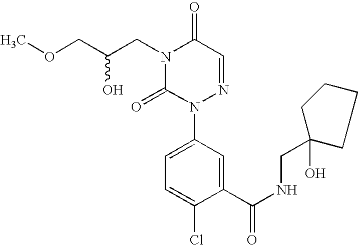 | 2-Chloro-N-(1-hydroxy- cyclopentylmethyl)-5-[4- (2-bydroxy-3-methoxy- propyl)-3,5-dioxo-4,5- dihydro-3H-[1,2,4]triazin- 2-yl]-benzamide | 453.5 |
| 77 | 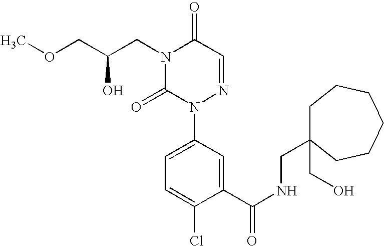 | 2-Chloro-5-[4-(2- hydroxy-3-methoxy- propyl)-3,5-dioxo-4,5- [1,2,4]triazin-2-yl]-N-(1- hydroxymethyl- cycloheptylmethyl)- benzamide | 495.4 |
| 78 | 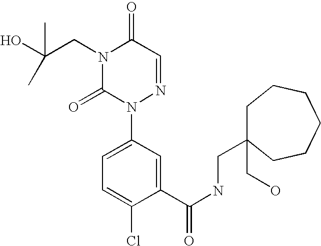 | 2-Chloro-N-(1- hydroxymethyl- cycloheptylmethyl)-5-[4- (2-hydroxy-2-methyl- propyl)-3,5-dioxo-4,5- dihydro-3H-[1,2,4]triazin- 2-yl]-benzamide | 479.4 |
| 79 | 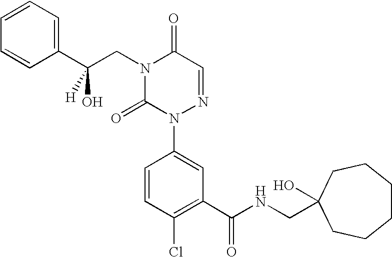 | 2-Chloro-N-(1-hydroxy- cycloheptylmethyl)-5-[4- (2-hydroxy-2-phenyl- ethyl)-3,5-dioxo-4,5- dihydro-3H-[1,2,4]triazin- 2-yl]-benzamide | 513.4 |
| 80 | 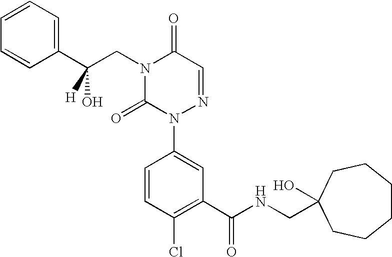 | 2-Chloro-N-(1-hydroxy- cycloheptylmethyl)-5-[4- (2-hydroxy-2-phenyl- ethyl)-3,5-dioxo-4,5- dihydro-3H-[1,2,4]triazin- 2-yl]-benzamide | 513.3 |
| 81 | 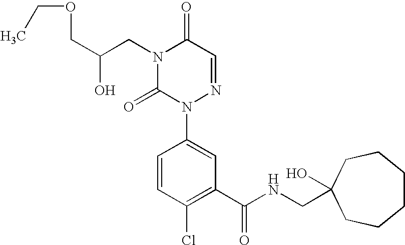 | 2-Chloro-5-[4-(3-ethoxy- 2-hydroxy-propyl)-3,5- dioxo-4,5-dihydro-3H- [1,2,4]triazin-2-yl]-N-(1- hydroxy- cycloheptylmethyl)- benzamide | 495.4 |
| 82 | 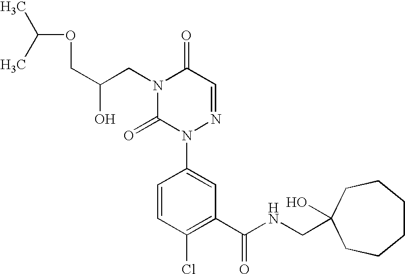 | 2-Chloro-N-(1-hydroxy- cycloheptylmethyl)-5-[4- (2-hydroxy-3-isopropoxy- propyl)-3,5-dioxo-4,5- dihydro-3H-[1,2,4]triazin- 2-yl]-benzamide | 509.4 |
| 83 | 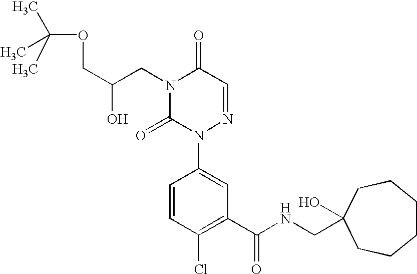 | 5-[4-(3-tert-Butoxy-2- hydroxy-propyl)-3,5- dioxo-4,5-dihydro-3H- [1,2,4]triazin-2-yl]-2- chloro-N-(1-hydroxy- cycloheptylmethyl)- benzamide | 523.4 |
| 84 | 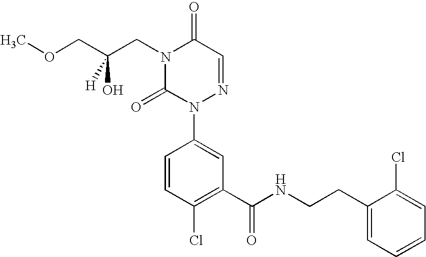 | 2-Chloro-N-[2-(2-chloro- phenyl)-ethyl]-5-[4-(2- hydroxy-3-methoxy- propyl)-3,5-dioxo-4,5- dihydro-3H-[1,2,4]triazin- 2-yl]-benzamide | 494.4 |
| 85 | 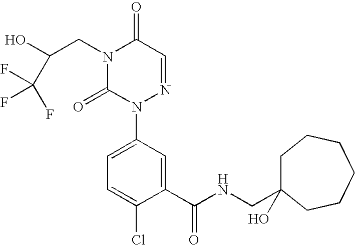 | 2-Chloro-5-[3,5-dioxo-4- (3,3,3-trifluoro-2- hydroxy-propyl)-4,5- dihydro-3H-[1,2,4]triazin- 2-yl]-N-(1-hydroxy- cycloheptylmethyl)- benzamide | 505.3 |
| 86 | 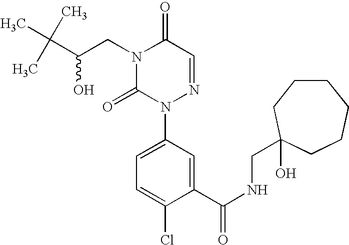 | 2-Chloro-N-(1-hydroxy- cycloheptylmethyl)-5-[4- (2-hydroxy-3,3-dimethyl- butyl)-3,5-dioxo-4,5- dihydro-3H-[1,2,4]triazin- 2-yl]-benzamide | 493.4 |
| 87 | 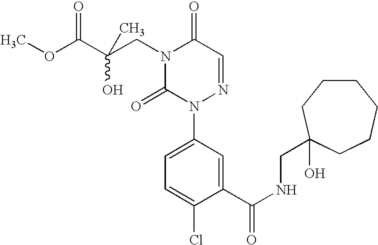 | 3-(2-{4-Chloro-3-[(1- hydroxy- cycloheptylmethyl)- carbamoyl]-phenyl}-3,5- dioxo-2,5-dihydro-3H- [1,2,4]triazin-4-yl)-2- hydroxy-2-methyl- propionic acid methyl ester | 509.4 |
| 88 | 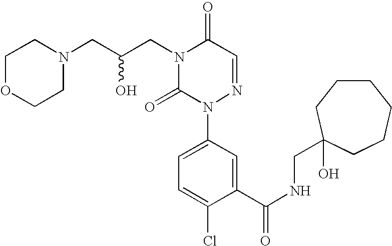 | 2-Chloro-N-(1-hydroxy- cycloheptylmethyl)-5-[4- (2-hydroxy-3-morpholin- 4-yl-propyl)-3,5-dioxo- 4,5-dihydro-3H- [1,2,4]triazin-2-yl]- benzamide | 536.4 |
| 89 | 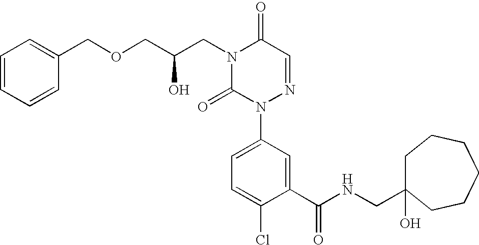 | 5-[4-(3-Benzyloxy-2- hydroxy-propyl)-3,5- dioxo-4,5-dihydro-3H- [1,2,4]triazin-2-yl]-2- chloro-N-(1-hydroxy- cycloheptylmethyl)- benzamide | 557.4 |
| 90 | 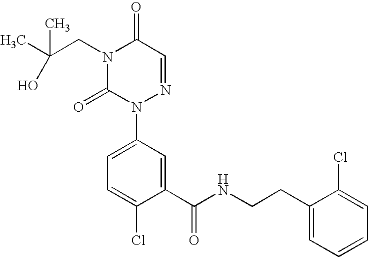 | 2-Chloro-N-[2-(2-chloro- hydroxy-2-methyl- propyl)-3,5-dioxo-4,5- dihydro-3H- [1,2,4]triazin-2-yl]- benzamide | 478.3 |
| 91 | 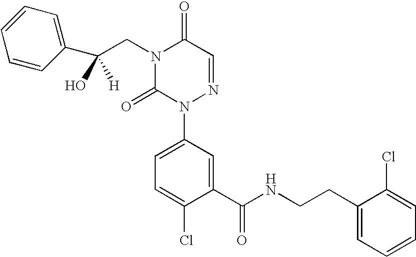 | 2-Chloro-N-[2-(2-chloro- phenyl)-ethyl]-5-[4-(2- hydroxy-2-phenyl-ethyl)- 3,5-dioxo-4,5-dihydro- 3H-[1,2,4]triazin-2-yl]- benzamide | 525.4 |
| 92 | 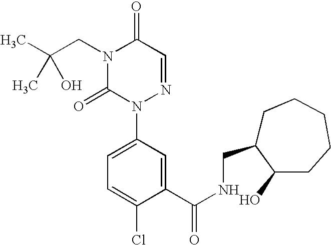 | 2-Chloro-N-(2-hydroxy- cycloheptylmethyl)-5-[4- propyl)-3,5-dioxo-4,5- dihydro-3H-[1,2,4]triazin- 2-yl]-benzamide | 465.4 |
| 93 | 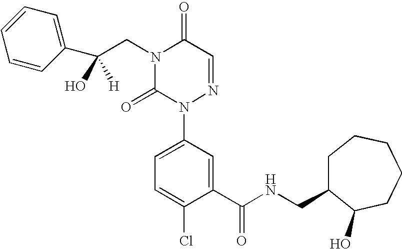 | 2-Chloro-N-(2-hydroxy- cycloheptylmethyl)-5-[4- (2-hydroxy-2-phenyl- ethyl)-3,5-dioxo-4,5- dihydro-3H-[1,2,4]triazin- 2-yl]-benzamide | 513.4 |
| 94 | 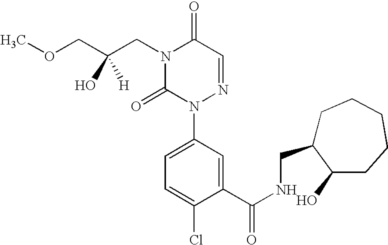 | 2-Chloro-N-(2-hydroxy- cycloheptylmethyl)-5-[4- (2-hydroxy-3-methoxy- propyl)-3,5-dioxo-4,5- dihydro-3H-[1,2,4]triazin- 2-yl]-benzamide | 481.4 |
| 95 | 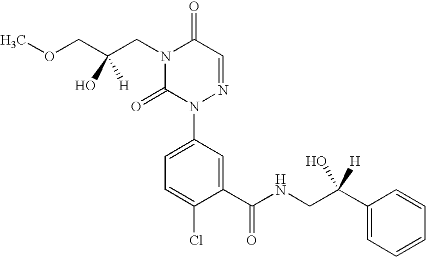 | 2-Chloro-5-[4-(2- hydroxy-3-methoxy- propyl)-3,5-dioxo-4,5- dihydro-3H-[1,2,4]triazin- 2-yl]-N-(2-hydroxy-2- phenyl-ethyl)-benzamide | 475.3 |
| 96 | 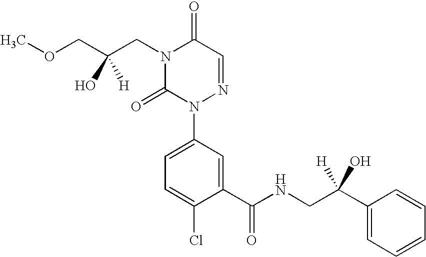 | 2-Chloro-5-[4-(2- hydroxy-3-methoxy- propyl)-3,5-dioxo-4,5- dihydro-3H-[1,2,4]triazin- 2-yl]-N-(2-hydroxy-2- phenyl-ethyl)-benzamide | 475.3 |
| 97 | 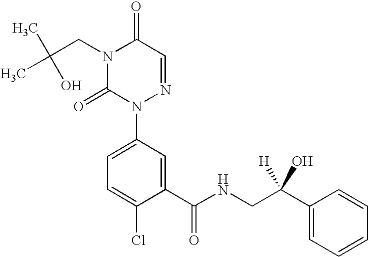 | 2-Chloro-5-[4-(2- hydroxy-2-methyl- propyl)-3,5-dioxo-4,5- dihydro-3H-[1,2,4]triazin- 2-yl]-N-(2-hydroxy-2- phenyl-ethyl)-benzamide | 459.3 |
| 98 | 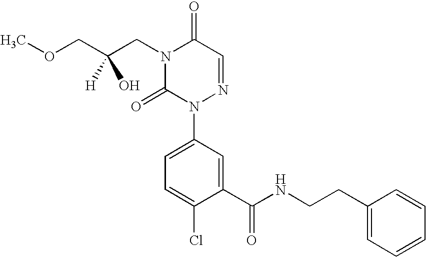 | 2-Chloro-5-[4-(2- hydroxy-3-methoxy- propyl)-3,5-dioxo-4,5- dihydro-3H-[1,2,4]triazin- 2-yl]-N-phenethyl- benzamide | 459.2 |
| 99 | 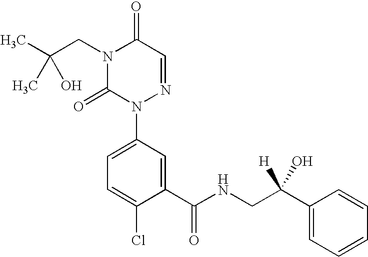 | 2-Chloro-5-[4-(2- hydroxy-2-methyl- propyl)-3,5-dioxo-4,5- dihydro-3H-[1,2,4]triazin- 2-yl]-N-(2-hydroxy-2- phenyl-ethyl)-benzamide | 459.3 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated herein by reference in their entireties.

What is claimed is:

1. A method of treatment of an IL-1 mediated disease in a mammal, the method comprising administering to a mammal in need thereof a pharmaceutically effective amount of a pharmaceutical agent selected from:
a) sulfasalazine;
b) a statin;
c) a glucocorticoid agent;
d) an inhibitor of p38 kinase;
e) an anti-IL-6-receptor antibody;
f) anakinra;
g) an anti-IL-1 monoclonal antibody;
h) an inhibitor of JAK3 protein tyrosine kinase;
i) a M-CSF monoclonal antibody; or
j) an anti-CD20 monoclonal antibody;
and a pharmaceutically effective amount of a compound of formula (I): 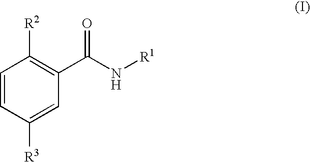
wherein R¹ is (C₁-C₆)alkyl, optionally substituted by (C₃-C₁₀)cycloalkyl, (C₆-C₁₀)aryl, (C₁-C₁₀)heterocyclyl, or (C₁-C₁₀)heteroaryl, wherein each of said (C₁-C₆)alkyl, (C₃-C₁₀)cycloalkyl, (C₆-C₁₀)aryl, (C₁-C₁₀)heterocyclyl, or (C₁-C₁₀)heteroaryl are optionally substituted by one to three suitable moieties independently selected from the group consisting of hydroxy, halogen, —CN, (C₁-C₆)alkyl, HO(C₁-C₆)alkyl-, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-;
R² is hydrogen, halogen, —CN, and (C₁-C₆)alkyl, wherein said (C₁-C₆)alkyl is optionally substituted by one to three moieties independently selected from halo, hydroxy, amino, —CN, (C₁-C₆)alkyl, (C₁-C₆)alkoxy, —CF₃, CF₃O—, (C₁-C₆)alkyl-NH—, [(C₁-C₆)alkyl]₂—N—, (C₁-C₆)alkyl-S—, (C₁-C₆)alkyl-(S═O)—, (C₁-C₆)alkyl-(SO₂)—, (C₁-C₆)alkyl-O—(C═O)—, formyl, (C₁-C₆)alkyl-(C═O)—, or (C₃-C₆)cycloalkyl; and
R³ is a suitably substituted nitrogen linked (C₁-C₁₀)heterocyclyl of the formula: 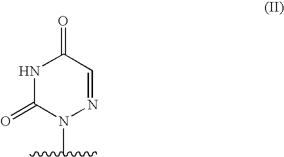
or the pharmaceutically acceptable salts or solvates or prodrugs thereof.

2. The method of claim 1 wherein the compound of formula (I) has the structure: 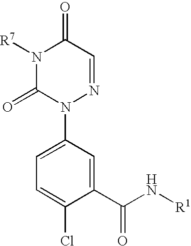
wherein R⁷ is as defined in claim 1 and R¹ is C₁-C₃ alkyl substituted by a C₃-C₈ or phenyl ring, the C₃-C₈ and phenyl rings being optionally substituted by from 1 to 4 substituents selected from the group of OH, halo, C₁-C₃ alkyl, C₁-C₃ alkoxy, or C₁-C₃ alkyl substituted by OH.

3. The method of claim 1 wherein the IL-1 mediated disease is selected from rheumatoid arthritis, osteoarthritis, Crohn's disease, chronic obstructive pulmonary disease, inflammatory bowel disease, Alzheimer's disease, psoriasis, psoriatic arthritis or atherosclerosis.

4. The method of claim 1 wherein the compound of formula (I) is selected from the group consisting of:
2-Chloro-N-(1-hydroxy-cyclohexylmethyl)-5-[4-(2-hydroxy-3-methoxy-propyl)-3,5-dioxo-4,5-dihydro-3H-[1,2,4]triazin-2-yl]-benzamide;
2-Chloro-5-[4-(2,3-dihydroxy-propyl)-3,5-dioxo-4,5-dihydro-3H-[1,2,4]triazin-2-yl]-N-(1-hydroxy-cyclohexylmethyl)-benzamide;
2-Chloro-N-(1-hydroxy-cycloheptylmethyl)-5-[4-(2-hydroxy-ethyl)-3,5-dioxo-4,5-dihydro-3H-[1,2,4]triazin-2-yl]-benzamide;
2-Chloro-5-[4-(2,3-dihydroxy-propyl)-3,5-dioxo-4,5-dihydro-3H-[1,2,4]triazin-2-yl]-N-(1-hydroxy-cycloheptylmethyl)-benzamide;
2-Chloro-5-(4-cyanomethyl-3,5-dioxo-4,5-dihydro-3H-[1,2,4]triazin-2-yl)-N-(1-hydroxy-cycloheptylmethyl)-benzamide;
2-Chloro-5-[4-(2-hydroxy-3-methoxy-propyl)-3,5-dioxo-4,5-dihydro-3H-[1,2,4]triazin-2-yl]-N-(1-hydroxymethyl-cycloheptylmethyl)-benzamide;
2-Chloro-5-[4-(2-cyano-ethyl)-3,5-dioxo-4,5-dihydro-3H-[1,2,4]triazin-2-yl]-N-(1-hydroxy-cycloheptylmethyl)-benzamide;
N-(1-Hydroxy-cycloheptylmethyl)-5-[4-(2-hydroxy-ethyl)-3,5-dioxo-4,5-dihydro-3H-[1,2,4]triazin-2-yl]-2-methyl-benzamide;
2-Chloro-5-[4-(2,3-dihydroxy-propyl)-3,5-dioxo-4,5-dihydro-3H-[1,2,4]triazin-2-yl]-N-(1-hydroxy-cyclohexylmethyl)-benzamide;
2-Chloro-N-(1-hydroxy-cycloheptylmethyl)-5-[4-(2-hydroxy-2-methyl-propyl)-3,5-dioxo-4,5-dihydro-3H-[1,2,4]triazin-2-yl]-benzamide;
2-Chloro-N-(1-hydroxy-cyclooctylmethyl)-5-[4-(2-hydroxy-3-methoxy-propyl)-3,5-dioxo-4,5-dihydro-3H-[1,2,4]triazin-2-yl]-benzamide;
2-Chloro-N-(1-hydroxy-cycloheptylmethyl)-5-[4-(2-hydroxy-2-phenyl-ethyl)-3,5-dioxo-4,5-dihydro-3H-[1,2,4]triazin-2-yl]-benzamide;
2-Chloro-5-[3,5-dioxo-4-(3,3,3-trifluoro-2-hydroxy-propyl)-4,5-dihydro-3H-[1,2,4]triazin-2-yl]-N-(1-hydroxy-cycloheptylmethyl)-benzamide;
2-Chloro-5-[4-(2-hydroxy-3-methoxy-propyl)-3,5-dioxo-4,5-dihydro-3H-[1,2,4]triazin-2-yl]-N-(2-hydroxy-2-phenyl-ethyl)-benzamide;
5-(4-Carbamoylmethyl-3,5-dioxo-4,5-dihydro-3H-[1,2,4]triazin-2-yl)-2-chloro-N-(1-hydroxy-cycloheptylmethyl)-benzamide;
2-Chloro-N-(1-hydroxy-cycloheptylmethyl)-5-[4-(2-methoxy-ethyl)-3,5-dioxo-4,5-dihydro-3H-[1,2,4]triazin-2-yl]-benzamide;
5-[4-(2,3-Dihydroxy-propyl)-3,5-dioxo-4,5-dihydro-3H-[1,2,4]triazin-2-yl]-N-(1-hydroxy-cycloheptylmethyl)-2-methyl-benzamide;
5-[4-(3-Amino-2-hydroxy-propyl)-3,5-dioxo-4,5-dihydro-3H-[1,2,4]triazin-2-yl]-2-chloro-N-(1-hydroxy-cycloheptylmethyl)-benzamide; and
2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide.

5. A method of treatment in a mammal of an IL-1 mediated disease selected from rheumatoid arthritis, osteoarthritis, juvenile arthritis, Crohn's disease, chronic obstructive pulmonary disease, inflammatory bowel disease, Alzheimer's disease, psoriasis, psoriatic arthritis or atherosclerosis, the method comprising administering to a mammal in need thereof a pharmaceutically effective amount of sulfasalazine, or a pharmaceutically acceptable salt form thereof, and a pharmaceutically effective amount of a compound of formula (I): 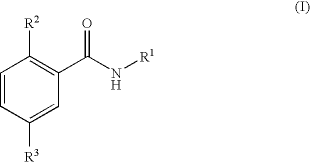
wherein R¹ is (C₁-C₆)alkyl, optionally substituted by (C₃-C₁₀)cycloalkyl, (C₆-C₁₀)aryl, (C₁-C₁₀)heterocyclyl, or (C₁-C₁₀)heteroaryl, wherein each of said (C₁-C₆)alkyl, (C₃-C₁₀)cycloalkyl, (C₆-C₁₀)aryl, (C₁-C₁₀)heterocyclyl, or (C₁-C₁₀)heteroaryl are optionally substituted by one to three suitable moieties independently selected from the group consisting of hydroxy, halogen, —CN, (C₁-C₆)alkyl, HO(C₁-C₆)alkyl, (C₁-C₆)alkyl-NH(C═O)—, NH₂(C═O)—, (C₁-C₆)alkoxy, or (C₃-C₁₀)cycloalkyl, wherein said (C₃-C₁₀)cycloalkyl is optionally substituted by one or more moieties selected from halogen, or (C₁-C₆)alkyl-;
R² is hydrogen, halogen, —CN, and (C₁-C₆)alkyl, wherein said (C₁-C₆)alkyl is optionally substituted by one to three suitable moieties, independently selected from the group consisting of halo, hydroxy, amino, —CN, (C₁-C₆)alkyl, (C₁-C₆)alkoxy, —CF₃, CF₃O—, (C₁-C₆)alkyl-NH—, [(C₁-C₆)alkyl]₂—N—, (C₁-C₆)alkyl-S—, (C₁-C₆)alkyl-(S═O)—, (C₁-C₆)alkyl-(SO₂)—, (C₁-C₆)alkyl-O—(C═O)—, formyl, (C₁-C₆)alkyl-(C═O)—, and (C₃-C₆)cycloalkyl; and
R³ is a suitably substituted nitrogen linked (C₁-C₁₀)heterocyclyl of the formula: 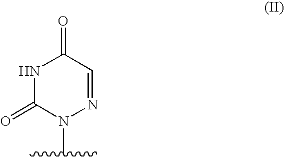
or the pharmaceutically acceptable salts or solvates or prodrugs thereof.

6. The method of claim 4 wherein compound of formula (I) is 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide.

7. A method of treatment of rheumatoid arthritis in a mammal, the method comprising administering to a mammal in need thereof a pharmaceutically effective amount of sulfasalazine, or a pharmaceutically acceptable salt form thereof, and a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide.

8. A pharmaceutical composition comprising a pharmaceutically effective amount of sulfasalazine, or a pharmaceutically acceptable salt form thereof, a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide and one or more pharmaceutically acceptable carriers or excipients.

9. A kit comprising a pharmaceutical formulation containing a pharmaceutically effective amount of sulfasalazine, or a pharmaceutically acceptable salt form thereof, and a pharmaceutical formulation containing a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide.

10. A method of treatment of rheumatoid arthritis in a mammal, the method comprising administering to a mammal in need thereof a pharmaceutically effective amount of atorvastatin, or a pharmaceutically acceptable salt form thereof, and a pharmaceutically effective amount of 2-Chloro-N-(1-hydroxy-cycloheptylmethyl)-5-[4-(2-hydroxy-3-methoxy-propyl)-3,5-dioxo-4,5-dihydro-3H-[1,2,4]triazin-2-yl]-benzamide.
